(19) United States
(12) Patent Application Publication
(10) Pub. No.: US 2018/0000645 A1
(43) Pub. Date: Jan. 4, 2018

(54) REUSABLE LASER PROBE WITH SINGLE-USE OPTIC FIBER

(71) Applicant: Katalyst Surgical, LLC, Chesterfield, MO (US)

(72) Inventors: Gregg D Scheller, Wildwood, MO (US); Eric J Bass, Webster Groves, MO (US); Matthew N Yates, High Ridge, MO (US); Christina D McNiff, Troy, MO (US); Steven G Scheller, Chesterfield, MO (US); Daniel J Wiener, St. Charles, MO (US)

(73) Assignee: Katalyst Surgical, LLC, Chesterfield, MO (US)

(21) Appl. No.: 15/629,480

(22) Filed: Jun. 21, 2017

Related U.S. Application Data
(60) Provisional application No. 62/357,444, filed on Jul. 1, 2016.

(51) Int. Cl. A61F 9/008; G02B 6/42

(52) U.S. Cl. CPC A61F 9/00823; G02B 6/4243; G02B 6/4296

(57) ABSTRACT

A reusable laser probe with single-use optic fiber may include a reusable handle, an optic fiber fixture, and a single-use optic fiber. The single-use optic fiber may include an optic fiber having an optic fiber distal end and an optic fiber proximal end. The optic fiber may be disposed in a first transitory connector having a first transitory connector distal end and a first transitory connector proximal end wherein the optic fiber distal end extends a fixed distance from the transitory connector distal end. The optic fiber may be disposed in a second transitory connector having a second transitory connector distal end and a second transitory connector proximal end wherein the optic fiber proximal end extends a fixed distance from the second transitory connector distal end. The first transitory connector may be inserted in the reusable handle and the second transitory connector may be inserted in the optic fiber fixture.

### CROSS-REFERENCE TO RELATED APPLICATIONS

This application claims the benefit of U.S. Provisional Application No. 62/357,444 filed Jul. 1, 2016.

### FIELD OF THE INVENTION

The present disclosure relates to a surgical instrument, and, more particularly, to a reusable laser probe with single-use optic fiber.

### BACKGROUND OF THE INVENTION

A wide variety of ophthalmic procedures require a laser energy source. For example, ophthalmic surgeons may use laser photocoagulation to treat proliferative retinopathy. Proliferative retinopathy is a condition characterized by the development of abnormal blood vessels in the retina that grow into the vitreous humor. Ophthalmic surgeons may treat this condition by energizing a laser to cauterize portions of the retina to prevent the abnormal blood vessels from growing and hemorrhaging. Typically, treatments are performed using a disposable, single-use laser probe connected to a laser surgical machine by an optical fiber. Unfortunately, use of disposable, single-use laser probes increases treatment costs because a new laser probe is required for each surgical treatment. Accordingly, there is a need for a laser probe that may be safely used to perform more than one surgical procedure.

### BRIEF SUMMARY OF THE INVENTION

The present disclosure provides a reusable laser probe with single-use optic fiber. In one or more embodiments, a reusable laser probe with single-use optic fiber may comprise a reusable handle, an optic fiber fixture, and a single-use optic fiber. Illustratively, the single-use optic fiber may comprise an optic fiber having an optic fiber distal end and an optic fiber proximal end. In one or more embodiments, the optic fiber may be disposed in a first transitory connector having a first transitory connector distal end and a first transitory connector proximal end. Illustratively, the optic fiber may be disposed in the first transitory connector wherein the optic fiber distal end extends a fixed distance from the transitory connector distal end. In one or more embodiments, the optic fiber may be disposed in a second transitory connector having a second transitory connector distal end and a second transitory connector proximal end. Illustratively, the optic fiber may be disposed in the second transitory connector wherein the optic fiber proximal end extends a fixed distance from the second transitory connector distal end. In one or more embodiments, the first transitory connector may be inserted in the reusable handle and the second transitory connector may be inserted in the optic fiber fixture.

### BRIEF DESCRIPTION OF THE DRAWINGS

The above and further advantages of the present invention may be better understood by referring to the following description in conjunction with the accompanying drawings in which like reference numerals indicate identical or functionally similar elements:

FIGS. 1A and 1B are schematic diagrams illustrating a transitory connector;

FIG. 2 is a schematic diagram illustrating an exploded view of a single-use optic fiber assembly;

FIGS. 3A and 3B are schematic diagrams illustrating an assembled single-use optic fiber;

FIG. 4 is a schematic diagram illustrating an exploded view of an optic fiber fixture assembly;

FIGS. 5A and 5B are schematic diagrams illustrating an assembled optic fiber fixture;

FIGS. 6A and 6B are schematic diagrams illustrating a handle facilitating sleeve;

FIGS. 7A and 7B are schematic diagrams illustrating a hypodermic tube;

FIGS. 8A and 8B are schematic diagrams illustrating an exploded view of a reusable handle assembly;

FIGS. 9A and 9B are schematic diagrams illustrating an assembled reusable handle;

FIGS. 10A and 10B are schematic diagrams illustrating an assembled reusable laser probe with single-use optic fiber;

FIGS. 11A, 11B, and 11C are schematic diagrams illustrating an optic fiber support mechanism;

FIG. 12 is a schematic diagram illustrating a reusable steerable handle assembly;

FIGS. 13A, 13B, 13C, and 13D are schematic diagrams illustrating an assembled reusable steerable handle;

FIGS. 14A an 14B are schematic diagrams illustrating an assembled reusable steerable laser probe with single-use optic fiber.

### DETAILED DESCRIPTION OF AN ILLUSTRATIVE EMBODIMENT

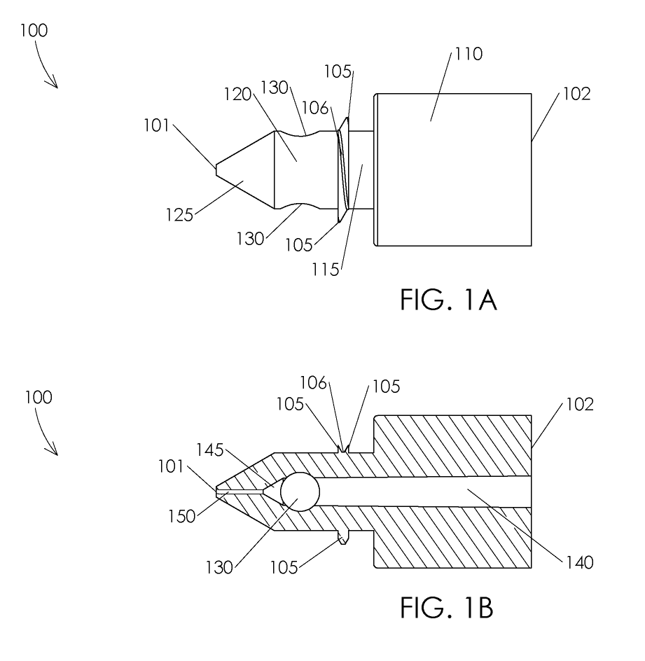

FIGS. 1A and 1B are schematic diagrams illustrating a transitory connector 100. FIG. 1A illustrates a side view of a transitory connector 100. FIG. 1B illustrates a cross-sectional view in a transverse plane of a transitory connector 100. In one or more embodiments, transitory connector 100 may comprise a transitory connector distal end 101 and a transitory connector proximal end 102. Illustratively, transitory connector 100 may comprise a major diameter 105 and a minor diameter 106. In one or more embodiments, transitory connector 100 may comprise a proximal base 110, a distal base 115, a nosecone base 120, and a nosecone 125. Illustratively, major diameter 105 may be disposed between distal base 115 and nosecone base 120. In one or more embodiments, minor diameter 106 may be disposed between distal base 115 and nosecone base 120. Illustratively, distal base 115 may be disposed between proximal base 110 and nosecone base 120. In one or more embodiments, nosecone base 120 may be disposed between distal base 115 and nosecone 125. Illustratively, transitory connector 100 may comprise an access lumen 130. In one or more embodiments, transitory connector 100 may comprise an inner bore 140. Illustratively, transitory connector 100 may comprise an inner bore distal taper 145. In one or more embodiments, transitory connector 100 may comprise an optic fiber housing 150. Illustratively, access lumen 130 may be disposed between inner bore 140 and inner bore distal taper 145. In one or more embodiments, inner bore distal taper 145 may be disposed between optic fiber housing 150 and access lumen 130. Illustratively, access lumen 130 may be disposed between optic fiber housing 150 and inner bore 140. In one or more embodiments, major diameter 105 may comprise a screw thread. Illustratively, minor diameter 106 may comprise a screw thread.

In one or more embodiments, transitory connector 100 may be manufactured from a material configured to deform if transitory connector 100 is sterilized in a medical autoclave, e.g., transitory connector 100 may be manufactured from a material configured to permanently deform if transitory connector 100 is sterilized in a medical autoclave. Illustratively, transitory connector 100 may be manufactured from a material having a melting point below a temperature parameter for a steam sterilization cycle, e.g., transitory connector 100 may be manufactured from a material having a melting point below a temperature parameter for a gravity-displacement steam sterilization cycle, a dynamic-air-removal steam sterilization cycle, etc. In one or more embodiments, transitory connector 100 may be manufactured from a material having a melting point below 140.0 degrees Fahrenheit. Illustratively, transitory connector 100 may be manufactured from a material having a melting point in a range of 158.0 to 212.0 degrees Fahrenheit, e.g., transitory connector 100 may be manufactured from a material having a melting point of 160.0 degrees Fahrenheit. In one or more embodiments, transitory connector 100 may be manufactured from a material having a melting point of less than 158.0 degrees Fahrenheit or greater than 212.0 degrees Fahrenheit. In one or more embodiments, transitory connector 100 may be manufactured from a material having a melting point below 250.0 degrees Fahrenheit. Illustratively, transitory connector 100 may be manufactured from a material having a melting point below 270.0 degrees Fahrenheit. In one or more embodiments, transitory connector 100 may be manufactured from a material having a melting point below 275.0 degrees Fahrenheit.

Illustratively, transitory connector 100 may be manufactured from a material configured to temporarily deform if transitory connector 100 is sterilized in a medical autoclave, e.g., transitory connector 100 may be manufactured from a material configured to absorb water in a medical autoclave. In one or more embodiments, an absorption of water may be configured to deform transitory connector 100, e.g., an absorption of water may be configured to cause transitory connector 100 to expand. Illustratively, transitory connector 100 may be manufactured from a porous material configured to facilitate a deformation of transitory connector 100 if transitory connector 100 is sterilized in a medical autoclave. In one or more embodiments, transitory connector 100 may be manufactured with one or more cavities configured to facilitate a deformation of transitory connector 100 if transitory connector 100 is sterilized in a medical autoclave. Illustratively, transitory connector 100 may be manufactured from any suitable material, e.g., polymers, metals, metal alloys, etc., or from any combination of suitable materials. In one or more embodiments, transitory connector 100 may be manufactured by a 3D printing process. For example, transitory connector 100 may be manufactured by selective laser sintering, selective heat sintering, selective laser melting, electron-beam melting, direct metal laser sintering, electron beam freeform fabrication, etc. Illustratively, transitory connector 100 may be manufactured by injection molding. In one or more embodiments, transitory connector 100 may be manufactured by additive manufacturing.

In one or more embodiments, transitory connector 100 may be manufactured from poly(acrylamide), poly(acrylic acid), poly(adipic anhydride), poly(7-aminoenanthic acid), poly(12-aminolauric acid), poly(11-aminoundecanoic acid), poly(azelaic anhydride), poly[1,3-butadiene(1,4-)-alt-methacrylonitrile], poly[1,3-butadiene(1,4-)-alt-methyl methacrylate], poly(butadiene oxide), poly(caprylaldehyde), poly(1,4-cyclohexylenedimethylene azelate), poly(1,4-cyclohexylenedimethylene dodecanedioate), poly(1,4-cyclohexylenedimethylene glutarate), poly(1,4-cyclohexylenedimethylene p-phenylenediacetate), poly(1,4-cyclohexylenedimethylene pimelate), poly(1,4-cyclohexylenedimethylene sebacate), poly(1,4-cyclohexylenedimethylene suberate), poly(cyclohexylidenethiohexamethylene sulfide), poly(cyclopropylenedimethylene piperazinediurethane), poly(cyclopropylidenedimethylene oxide), poly(decamethylene), poly(decamethylene carbonate), poly[(decamethylenedioxy)-dihexamethylene oxide], poly(decamethylene disulfide), poly(decamethylenedithioethylene disulfide), poly(decamethylenedithiohexamethylene disulfide), poly(decamethylene dithioladipate), poly(decamethylenedithiotetramethylene disulfide), poly(decamethylene pimelate), poly(decamethylene fumaramide), poly(decamethylene glutaramide), poly(decamethylene isophthalate), poly(decamethylene malonate), poly(decamethylene oxydiacetate), poly(decamethyleneoxymethylene oxide), poly(decamethylene succinate), poly(decamethylene sulfide), poly(decamethylene thiodivalerate), poly(decamethylenethiohexamethylene sulfide), poly(divinylbenzal), poly(dodecamethylene), poly(dodecanedioic anhydride), poly(eicosamethylene adipate), poly(eicosamethylene azelate), poly(eicosamethylene glutarate), poly(eicosamethylene isophthalate), poly(eicosamethylene malonate), poly(eicosamethylene oxalate), poly(eicosamethylene oxydiacetate), poly(eicosamethylene phthalate), poly(eicosamethylene pimelate), poly(eicosamethylene sebacate), poly(eicosamethylene suberate), poly(eicosamethylene succinate), poly(eicosamethylene thiodivalerate), poly[ethylene p-(carboxyphenoxy)-butyrate], poly[ethylene p-(carboxyphenoxy)-caproate], poly[ethylene p-(carboxyphenoxy)-heptanoate], poly[ethylene p-(carboxyphenoxy)-undecanoate], poly[ethylene p-(carboxyphenoxy)-valerate], poly(ethylene 2,2′-dibenzoate), poly[(ethylenedioxy)-diethylene 2,2′-dibenzoate], poly(ethylene 2,2′-dibenzoate), poly[(ethylenedioxy)-diethylene 3,3′-dibenzoate], poly[(ethylenedioxy)-diethylene isophthalate], poly[(ethylenedioxy)-diethylene sebacate], poly[(ethylenedioxy)-diethylene thiodivalerate], poly(ethylene disiloxanylenedipropionamide), poly[(ethylenedithio)-diacetic anhydride], poly[(ethylenedithio)-dipropionic anhydride], poly(ethylene dithionisophthalate), poly(ethelene dithiotetramethylene disulfide), poly(ethylene fumaramide), poly(ethylene glutarate), poly(ethylene 2,4-hexadienediamide), poly(ethylene phthalate), poly(ethylene sulfonyldivalerate), poly(ethylene terephthalate), poly(heptamethylene), poly(hexamethylene azelate), poly(hexamethylene carbonate), poly[hexamethylene p-(carboxyphenoxy)-acetate], poly[hexamethylene p-(carboxyphenoxy)-caproate], poly[hexamethylene p-(carboxyphenoxy)-undecanoate], poly[hexamethylene p-(carboxyphenoxy)-valerate], poly(hexamethylene isophthalate), poly[hexamethylene (methylene-2,5-tetrahydrofuran)-dicarboxamide], poly(hexamethylene octadecanediamide), poly(hexamethylene oxydiacetate), poly(hexamethylene 4,4′-oxydibenzoate), poly(hexamethylene pimelate), poly(hexamethylene succinate), poly(hexamethylene thiodivalerate), poly(hexamethylenethiooentamethylene sulfide), poly(hexamethylenethiotetramethylene sulfide), poly(hexenamer), etc. Illustratively, transitory connector 100 may be manufactured from any substituted polymers of poly(acrylamide), poly(acrylic acid), poly(adipic anhydride), poly(7-aminoenanthic acid), poly(12-aminolauric acid), poly(11-aminoundecanoic acid), poly(azelaic anhydride), poly[1,3-butadiene(1,4-)-alt-methacrylonitrile], poly[1,3-butadiene(1,4-)-alt-methyl methacrylate], poly(butadiene oxide), poly(capryl aldehyde), poly(1,4-cyclohexylenedimethylene azelate), poly(1,4-cyclohexylenedimethylene dodecanedioate), poly(1,4-cyclohexylenedimethylene glutarate), poly(1,4-cyclohexylenedimethylene p-phenylenediacetate), poly(1,4-cyclohexylenedimethylene pimelate), poly(1,4-cyclohexylenedimethylene sebacate), poly(1,4-cyclohexylenedimethylene suberate), poly(cyclohexylidenethiohexamethylene sulfide), poly(cyclopropylenedimethylene piperazinediurethane), poly(cyclopropylidenedimethylene oxide), poly(decamethylene), poly(decamethylene carbonate), poly[(decamethylenedioxy)-dihexamethylene oxide], poly(decamethylene disulfide), poly(decamethylenedithioethylene disulfide), poly(decamethylenedithiohexamethylene disulfide), poly(decamethylene dithioladipate), poly(decamethylenedithiotetramethylene disulfide), poly(decamethylene pimelate), poly(decamethylene fumaramide), poly(decamethylene glutaramide), poly(decamethylene isophthalate), poly(decamethylene malonate), poly(decamethylene oxydiacetate), poly(decamethyleneoxymethylene oxide), poly(decamethylene succinate), poly(decamethylene sulfide), poly(decamethylene thiodivalerate), poly(decamethylenethiohexamethylene sulfide), poly(divinylbenzal), poly(dodecamethylene), poly(dodecanedioic anhydride), poly(eicosamethylene adipate), poly(eicosamethylene azelate), poly(eicosamethylene glutarate), poly(eicosamethylene isophthalate), poly(eicosamethylene malonate), poly(eicosamethylene oxalate), poly(eicosamethylene oxydiacetate), poly(eicosamethylene phthalate), poly(eicosamethylene pimelate), poly(eicosamethylene sebacate), poly(eicosamethylene suberate), poly(eicosamethylene succinate), poly(eicosamethylene thiodivalerate), poly[ethylene p-(carboxyphenoxy)-butyrate], poly[ethylene p-(carboxyphenoxy)-caproate], poly[ethylene p-(carboxyphenoxy)-heptanoate], poly[ethylene p-(carboxyphenoxy)-undecanoate], poly[ethylene p-(carboxyphenoxy)-valerate], poly(ethylene 2,2′-dibenzoate), poly[(ethylenedioxy)-diethylene 2,2′-dibenzoate], poly(ethylene 2,2′-dibenzoate), poly[(ethylenedioxy)-diethylene 3,3′-dibenzoate], poly[(ethylenedioxy)-diethylene isophthalate], poly[(ethylenedioxy)-diethylene sebacate], poly[(ethylenedioxy)-diethylene thiodivalerate], poly(ethylene disiloxanylenedipropionamide), poly[(ethylenedithio)-diacetic anhydride], poly[(ethylenedithio)-dipropionic anhydride], poly(ethylene dithionisophthalate), poly(ethelene dithiotetramethylene disulfide), poly(ethylene fumaramide), poly(ethylene glutarate), poly(ethylene 2,4-hexadienediamide), poly(ethylene phthalate), poly(ethylene sulfonyldivalerate), poly(ethylene terephthalate), poly(heptamethylene), poly(hexamethylene azelate), poly(hexamethylene carbonate), poly[hexamethylene p-(carboxyphenoxy)-acetate], poly[hexamethylene p-(carboxyphenoxy)-caproate], poly[hexamethylene p-(carboxyphenoxy)-undecanoate], poly[hexamethylene p-(carboxyphenoxy)-valerate], poly(hexamethylene isophthalate), poly[hexamethylene (methylene-2,5-tetrahydrofuran)-dicarboxamide], poly(hexamethylene octadecanediamide), poly(hexamethylene oxydiacetate), poly(hexamethylene 4,4′-oxydibenzoate), poly(hexamethylene pimelate), poly(hexamethylene succinate), poly(hexamethylene thiodivalerate), poly(hexamethylenethiooentamethylene sulfide), poly(hexamethylenethiotetramethylene sulfide), poly(hexenamer), etc.

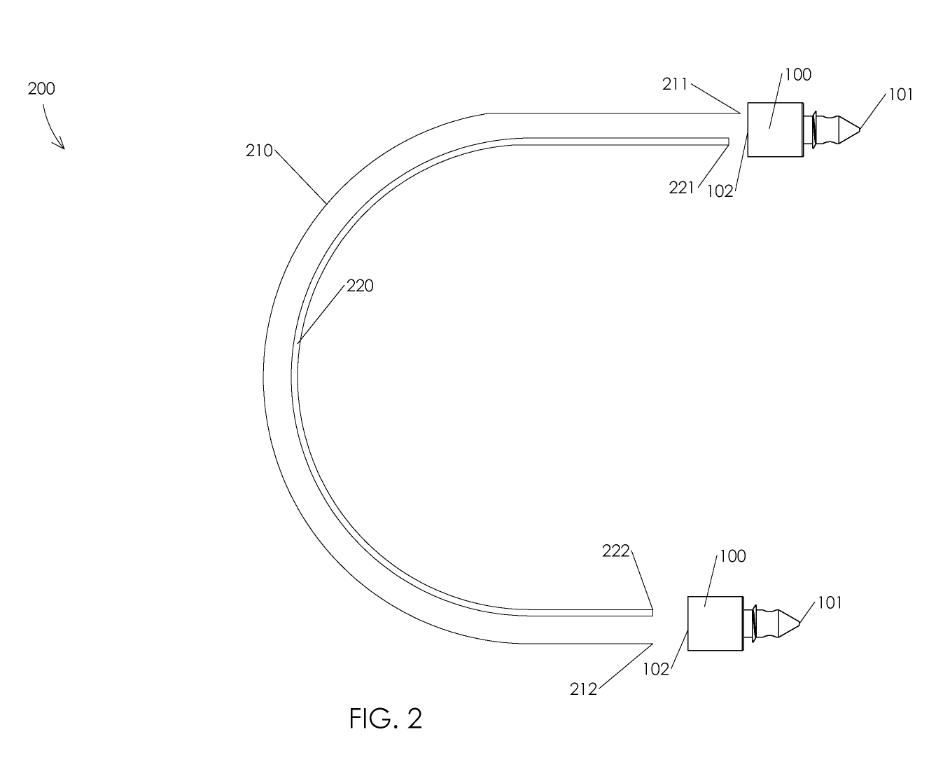

FIG. 2 is a schematic diagram illustrating an exploded view of a single-use optic fiber assembly 200. Illustratively, a single-use optic fiber assembly 200 may comprise a first transitory connector 100, an optic fiber 210, jacketing 220, and a second transitory connector 100. In one or more embodiments, optic fiber 210 may comprise an optic fiber distal end 211 and an optic fiber proximal end 212. Illustratively, optic fiber 210 may be configured to transmit laser light. In one or more embodiments, jacketing 220 may comprise a jacketing distal end 221 and a jacketing proximal end 222. Illustratively, jacketing 220 may be manufactured from any suitable material, e.g., polymers, metals, metal alloys, etc., or from any combination of suitable materials.

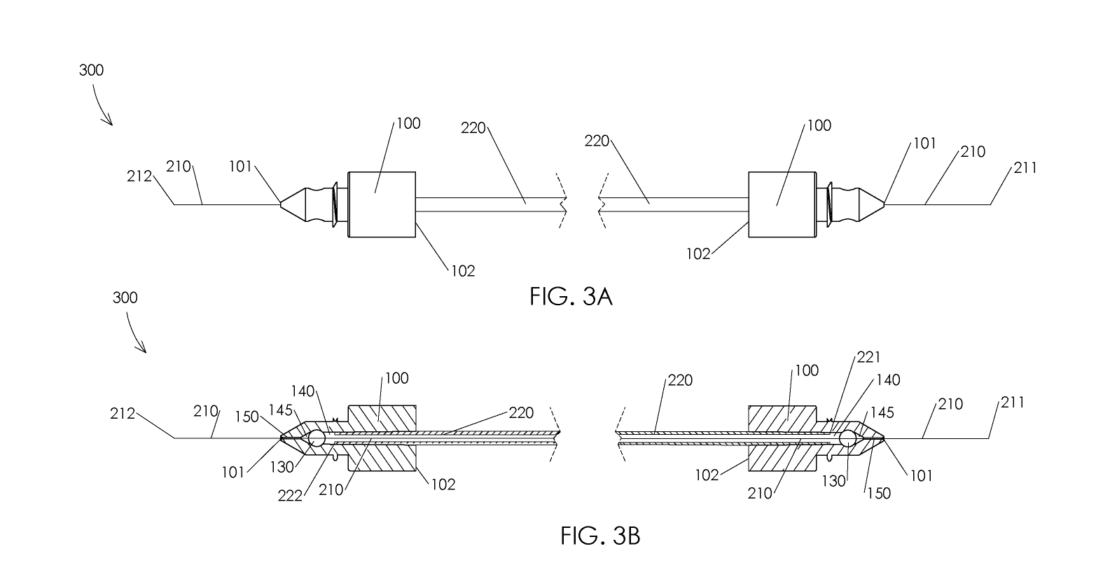

FIGS. 3A and 3B are schematic diagrams illustrating an assembled single-use optic fiber 300. FIG. 3A illustrates a side view of an assembled single-use optic fiber 300. FIG. 3B illustrates a cross-sectional view in a transverse plane of an assembled single-use optic fiber 300. Illustratively, optic fiber proximal end 212 may extend a distance from first transitory connector distal end 101. In one or more embodiments, optic fiber distal end 211 may extend a distance from second transitory connector distal end 101. Illustratively, optic fiber 210 may be disposed within jacketing 220 wherein optic fiber distal end 211 extends a distance from jacketing distal end 221 and wherein optic fiber proximal end 212 extends a distance from jacketing proximal end 222. In one or more embodiments, a portion of jacketing 220 may be disposed in a portion of first transitory connector 100, e.g., jacketing proximal end 222 may be disposed in inner bore 140. Illustratively, a portion of jacketing 220 may be fixed in a portion of first transitory connector 100, e.g., a portion of jacketing 220 may be fixed in a portion of first transitory connector 100 by an adhesive, a friction fit, a crimp, a tie, a weld, etc. In one or more embodiments, a portion of jacketing 220 may be disposed in a portion of second transitory connector 100, e.g., jacketing distal end 221 may be disposed in inner bore 140. Illustratively, a portion of jacketing 220 may be fixed in a portion of second transitory connector 100, e.g., a portion of jacketing 220 may be fixed in a portion of second transitory connector 100 by an adhesive, an epoxy, a friction fit, a crimp, a tie, a weld, etc.

In one or more embodiments, a portion of optic fiber 210 may be disposed in first transitory connector 100 wherein optic fiber 210 is disposed in inner bore 140, access lumen 130, inner bore distal taper 145, and optic fiber housing 150. Illustratively, a portion of optic fiber 210 may be fixed in a portion of first transitory connector 100, e.g. a portion of optic fiber 210 may be fixed in optic fiber housing 150. In one or more embodiments, a portion of optic fiber 210 may be fixed in optic fiber housing 150, e.g., a portion of optic fiber 210 may be fixed in optic fiber housing 150 by an adhesive, an epoxy, a friction fit, a tie, a crimp, a weld, etc. Illustratively, a portion of optic fiber 210 may be fixed in first transitory connector 100 wherein optic fiber proximal end 212 extends a distance from first transitory connector distal end 101. In one or more embodiments, a portion of optic fiber 210 may be disposed in second transitory connector 100 wherein optic fiber 210 is disposed in inner bore 140, access lumen 130, inner bore distal taper 145, and optic fiber housing 150. Illustratively, a portion of optic fiber 210 may be fixed in a portion of second transitory connector 100, e.g. a portion of optic fiber 210 may be fixed in optic fiber housing 150. In one or more embodiments, a portion of optic fiber 210 may be fixed in optic fiber housing 150, e.g., a portion of optic fiber 210 may be fixed in optic fiber housing 150 by an adhesive, an epoxy, a friction fit, a tie, a crimp, a weld, etc. Illustratively, a portion of optic fiber 210 may be fixed in second transitory connector 100 wherein optic fiber distal end 211 extends a distance from second transitory connector distal end 101. Illustratively, a distance that optic fiber distal end 211 extends from second transitory connector distal end 101 may be identical to a distance that optic fiber proximal end 212 extends from first transitory connector distal end 101. In one or more embodiments, a distance that optic fiber distal end 211 extends from second transitory connector distal end 101 and a distance that optic fiber proximal end 212 extends from first transitory connector distal end 101 may be configured to allow first transitory connector 100 and second transitory connector 100 to be interchangeable.

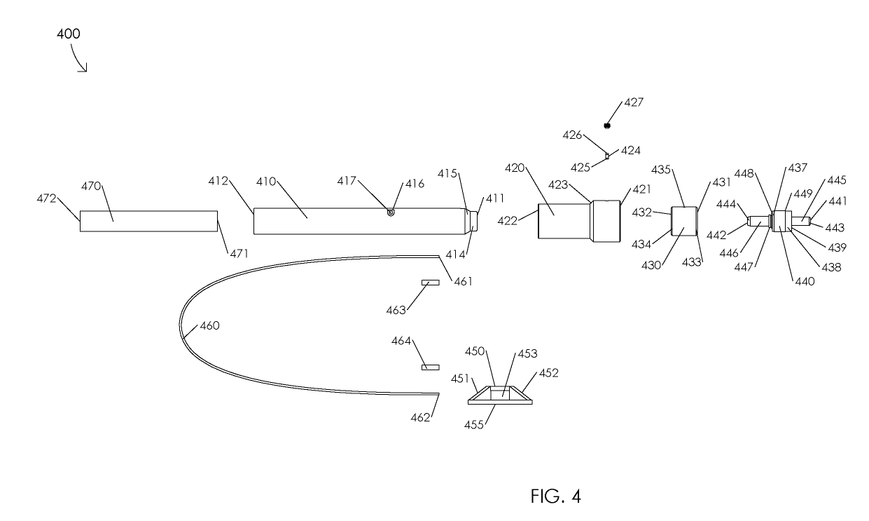

FIG. 4 is a schematic diagram illustrating an exploded view of an optic fiber fixture assembly 400. Illustratively, an optic fiber fixture assembly 400 may comprise a fixture base 410, a machine connector housing 420, an electrical element 424, a fixation mechanism 427, an extender 430, a machine connector 440, a machine coupler 450, a lanyard cable 460, a distal fastener 463, a proximal fastener 464, and a fixture facilitating sleeve 470. In one or more embodiments, fixture base 410 may comprise a fixture base distal end 411 and a fixture base proximal end 412. Illustratively, fixture base 410 may comprise an extender interface 414, a fixture base distal taper 415, an indentation 416, and a lanyard cable guide 417. In one or more embodiments, lanyard cable guide 417 may be disposed in indentation 416. Illustratively, lanyard cable guide 417 may be disposed dorsally in fixture base 410. In one or more embodiments, machine connector housing 420 may comprise a machine connector housing distal end 421 and a machine connector housing proximal end 422. Illustratively, machine connector housing 420 may comprise a machine connector housing taper 423. In one or more embodiments, extender 430 may comprise an extender distal end 431 and an extender proximal end 432. Illustratively, extender 430 may comprise an extender distal taper 433 and an extender proximal taper 434. In one or more embodiments, extender 430 may comprise an electrical element housing 435. Illustratively, electrical element housing 435 may be configured to house electrical element 424. In one or more embodiments, electrical element 424 may comprise an electrical element inferior end 425 and an electrical element superior end 426.

In one or more embodiments, machine connector 440 may comprise a machine connector distal end 441 and a machine connector proximal end 442. Illustratively, machine connector 440 may comprise a machine connector distal taper 443, a machine connector proximal taper 444, a distal ferrule 445, and a proximal ferrule 446. In one or more embodiments, machine connector 440 may comprise a machine connector base 438. Illustratively, machine connector base 438 may comprise a machine connector base distal end 439 and a machine connector base proximal end 449. In one or more embodiments, machine connector 440 may comprise a retaining ring distal interface 437, a retaining ring proximal interface 447, and a retaining ring 448. Illustratively, retaining ring 448 may be disposed between retaining ring distal interface 437 and retaining ring proximal interface 447. In one or more embodiments, lanyard cable 460 may comprise a lanyard cable distal end 461 and a lanyard cable proximal end 462. Illustratively, machine coupler 450 may comprise a machine coupler inferior end 451 and a machine coupler superior end 452. In one or more embodiments, machine coupler 450 may comprise a machine coupler aperture 453. Illustratively, machine coupler 450 may comprise a machine interface 455.

In one or more embodiments, fixture facilitating sleeve 470 may comprise a fixture facilitating sleeve distal end 471 and a fixture facilitating sleeve proximal end 472. Illustratively, fixture facilitating sleeve 470 may be manufactured from a material configured to minimize a coefficient of friction between a portion of optic fiber 210 and a portion of fixture facilitating sleeve 470, e.g., fixture facilitating sleeve 470 may be manufactured from a self-lubricating thermoplastic material. In one or more embodiments, fixture facilitating sleeve 470 may be manufactured from a material wherein a coefficient of friction between a portion of optic fiber 210 and a portion of fixture facilitating sleeve 470 is in a range of 0.011 to 0.36, e.g., fixture facilitating sleeve 470 may be manufactured from a material wherein a coefficient of friction between a portion of optic fiber 210 and a portion of fixture facilitating sleeve 470 is 0.0311. Illustratively, fixture facilitating sleeve 470 may be manufactured from a material wherein a coefficient of friction between a portion of optic fiber 210 and a portion of fixture facilitating sleeve 470 is less than 0.011 or greater than 0.36. In one or more embodiments, fixture facilitating sleeve 470 may be manufactured from a fluorocarbon material, e.g., fixture facilitating sleeve 470 may be manufactured from a polytetrafluoroethylene material. Illustratively, fixture facilitating sleeve 470 may be manufactured from an acetal-based polytetrafluoroethylene material, e.g., fixture facilitating sleeve 470 may be manufactured from a turcite material. In one or more embodiments, fixture facilitating sleeve 470 may be manufactured from a material having a density in a range of 0.024 to 0.073 pounds per cubic inch, e.g., fixture facilitating sleeve 470 may be manufactured from a material having a density of 0.053 pounds per cubic inch. Illustratively, fixture facilitating sleeve 470 may be manufactured from a material having a density of less than 0.024 pounds per cubic inch or greater than 0.073 pounds per cubic inch. In one or more embodiments, fixture facilitating sleeve 470 may be manufactured from a material having a hardness in a range of 50 Shore D to 75 Shore D, e.g., fixture facilitating sleeve 470 may be manufactured from a material having a hardness of 61 Shore D. Illustratively, fixture facilitating sleeve 470 may be manufactured from a material having a hardness of less than 50 Shore D or greater than 75 Shore D. In one or more embodiments, optic fiber 210 may be manufactured from a material having a first hardness and fixture facilitating sleeve 470 may be manufactured from a material having a second hardness. Illustratively, the first hardness may be greater than the second hardness. In one or more embodiments, optic fiber 210 may be manufactured from a material having a first hardness, fixture facilitating sleeve 470 may be manufactured from a material having a second hardness, and transitory connector 100 may be manufactured from a material having a third hardness. Illustratively, the first hardness may be greater than the second hardness and the second hardness may be greater than the third hardness.

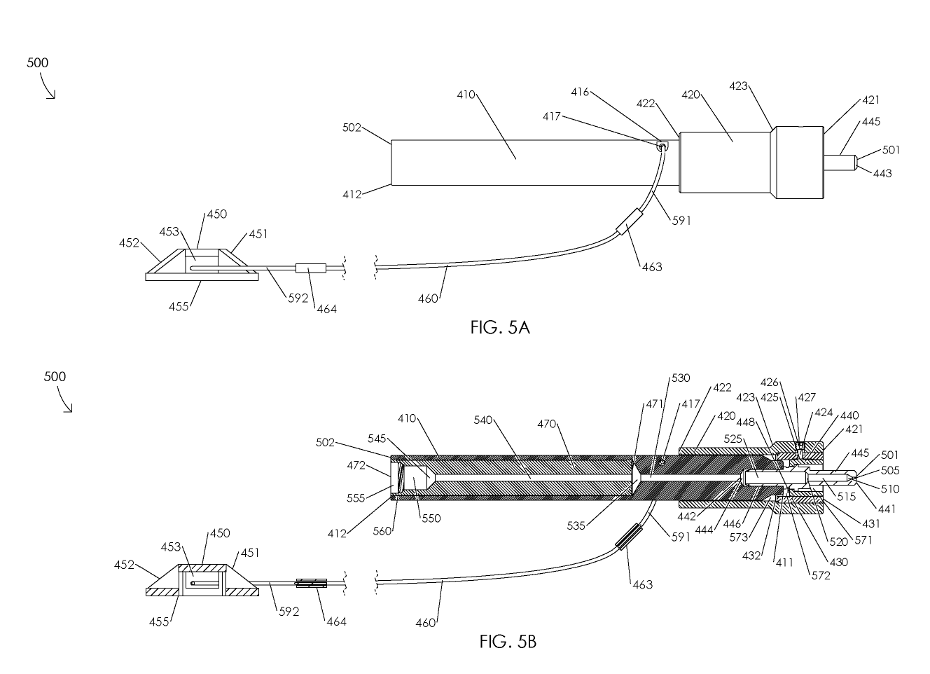

FIGS. 5A and 5B are schematic diagrams illustrating an assembled optic fiber fixture 500. FIG. 5A illustrates a side view of an assembled optic fiber fixture 500. FIG. 5B illustrates a cross-sectional view in a sagittal plane of an assembled optic fiber fixture 500. Illustratively, an assembled optic fiber fixture 500 may comprise an assembled optic fiber fixture distal end 501 and an assembled optic fiber fixture proximal end 502. In one or more embodiments, assembled optic fiber fixture 500 may comprise an optic fiber proximal end guide 505, an inner lumen distal taper 510, a machine connector distal inner lumen 515, an inner lumen proximal taper 520, a machine connector proximal inner lumen 525, a fixture base inner bore 530, a fixture facilitating sleeve housing 535, a fixture facilitating sleeve inner bore 540, a first transitory connector housing distal taper 545, a first transitory connector housing 550, a first transitory connector housing proximal chamber 555, and a first transitory connector housing threading 560.

Illustratively, fixture facilitating sleeve 470 may be disposed in a portion of fixture base 410, e.g., fixture facilitating sleeve 470 may be disposed in fixture facilitating sleeve housing 535. In one or more embodiments, fixture facilitating sleeve 470 may be disposed in a portion of fixture base 410 wherein fixture facilitating sleeve proximal end 472 is adjacent to fixture base proximal end 412, e.g., fixture facilitating sleeve 470 may be disposed in a portion of fixture base 410 wherein fixture facilitating sleeve proximal end 472 abuts fixture base proximal end 412. Illustratively, fixture facilitating sleeve 470 may be disposed in a portion of fixture base 410 wherein fixture facilitating sleeve inner bore 540 is aligned with fixture base inner bore 530, e.g., fixture facilitating sleeve 470 may be disposed in a portion of fixture base 410 wherein fixture facilitating sleeve inner bore 540 is collinear with fixture base inner bore 530. In one or more embodiments, fixture facilitating sleeve 470 may be disposed in a portion of fixture base 410 wherein fixture facilitating sleeve 470 is fixed in the portion of fixture base 410, e.g., fixture facilitating sleeve 470 may be fixed in a portion of fixture base 410 by an adhesive, an epoxy, a friction fit, a crimp, a tie, a weld, etc. In one or more embodiments, fixture facilitating sleeve 470 may be fixed in fixture facilitating sleeve housing 535, e.g., fixture facilitating sleeve 470 may be fixed in fixture facilitating sleeve housing 535 by an adhesive, an epoxy, a friction fit, a crimp, a tie, a weld, etc.

In one or more embodiments, lanyard cable 460 may comprise a distal loop 591 and a proximal loop 592. Illustratively, distal fastener 463 may be disposed over a portion of lanyard cable 460 wherein lanyard cable distal end 461 extends a distance from distal fastener 463. In one or more embodiments, lanyard cable distal end 461 may be threaded through lanyard cable guide 417 and into a portion of distal fastener 463 to form distal loop 591. Illustratively, distal fastener 463 may be configured to fix lanyard cable distal end 461 within distal fastener 463, e.g., distal fastener 463 may be configured to fix lanyard cable distal end 461 within distal fastener 463 by an adhesive, a crimp, a weld, a friction fit, etc. In one or more embodiments, proximal fastener 464 may be disposed over a portion of lanyard cable 460 wherein lanyard cable proximal end 462 extends a distance from proximal fastener 464. Illustratively, lanyard cable proximal end 462 may be threaded through machine coupler aperture 453 and into a portion of proximal fastener 464 to form proximal loop 592. In one or more embodiments, proximal fastener 464 may be configured to fix lanyard cable proximal end 462 within proximal fastener 464, e.g., proximal fastener 464 may be configured to fix lanyard cable proximal end 462 within proximal fastener 464 by an adhesive, an epoxy, a crimp, a weld, a friction fit, etc.

Illustratively, machine connector 440 may comprise a machine connector inner chamber 571. In one or more embodiments, distal ferrule 445 may extend a distance out from machine connector inner chamber 571. Illustratively, extender 430 may comprise an extender inner chamber 572. In one or more embodiments, machine connector 440 may be disposed in extender inner chamber 572. Illustratively, machine connector 440 may be disposed in extender 430, e.g., machine connector 440 may be disposed in extender 430 wherein machine connector distal end 441 may extend a distance from extender distal end 431 and wherein machine connector proximal end 442 may extend a distance from extender proximal end 432. In one or more embodiments, machine connector 440 may be fixed in extender 430, e.g., machine connector 440 may be fixed in extender 430 by an adhesive, a crimp, a weld, a friction fit, etc. Illustratively, machine connector housing 420 may comprise a machine connector housing inner chamber 573. In one or more embodiments, extender 430 may be disposed in machine connector housing inner chamber 573. Illustratively, extender 430 may be disposed in machine connector housing 420, e.g., extender 430 may be disposed in machine connector housing 420 wherein extender distal end 431 is disposed between machine connector housing distal end 421 and machine connector housing proximal end 422 and wherein extender proximal end 432 is disposed between machine connector housing distal end 421 and machine connector housing proximal end 422. In one or more embodiments, extender 430 may be disposed in machine connector housing 420 wherein machine connector distal end 441 extends a distance from machine connector housing distal end 421, e.g., extender may be disposed in machine connector housing 420 wherein machine connector proximal end 442 may be disposed between machine connector housing distal end 421 and machine connector housing proximal end 422. Illustratively, extender 430 may be fixed in machine connector housing 420, e.g., extender 430 may be fixed in machine connector housing 420 by an adhesive, an epoxy, a crimp, a weld, a friction fit, etc.

In one or more embodiments, fixture base 410 may be disposed in machine connector housing 420, e.g., fixture base 410 may be disposed in machine connector housing 420 wherein fixture base proximal end 412 extends a distance from machine connector housing proximal end 422 and wherein fixture base distal end 411 is disposed between machine connector housing distal end 421 and machine connector housing proximal end 422. Illustratively, fixture base 410 may be fixed in machine connector housing 420, e.g., fixture base 410 may be fixed in machine connector housing 420 by an adhesive, a crimp, a weld, a friction fit, etc. In one or more embodiments, fixture base 410 may be disposed in extender 430, e.g., fixture base 410 may be disposed in extender 430 wherein fixture base proximal end 412 extends a distance from extender proximal end 432 and wherein fixture base distal end 411 is disposed between extender distal end 431 and extender proximal end 432. Illustratively, fixture base 410 may be fixed in extender 430, e.g., fixture base 410 may be fixed in extender 430 by an adhesive, a crimp, a weld, a friction fit, etc. In one or more embodiments, machine connector 440 may be disposed in fixture base 410, e.g., machine connector 440 may be disposed in fixture base 410 wherein machine connector distal end 441 extends a distance from fixture base distal end 411 and wherein machine connector proximal end 442 is disposed between fixture base distal end 411 and fixture base proximal end 412. Illustratively, machine connector 440 may be fixed in fixture base 410, e.g., machine connector 440 may be fixed in fixture base 410 by an adhesive, an epoxy, a crimp, a weld, a friction fit, etc.

In one or more embodiments, electrical element 424 may be disposed in machine connector housing 420 and extender 430, e.g., electrical element may be disposed in electrical element housing 435. Illustratively, electrical element 424 may be fixed in electrical element housing 435, e.g., electrical element 424 may be fixed in electrical element housing 435 by an adhesive, an epoxy, a crimp, a weld, a friction fit, etc. In one or more embodiments, electrical element 424 may be disposed in machine connector housing 420 and extender 430 wherein electrical element inferior end 425 may be in contact with machine connector 440, e.g., electrical element 424 may be disposed in electrical element housing 435 wherein electrical element inferior end 425 may be in contact with machine connector 440. Illustratively, electrical element 424 may be electrically connected to machine connector 440. In one or more embodiments, electrical element 424 may be configured to convey data to a machine, e.g., electrical element 424 may be configured to convey data to a laser machine. Illustratively, electrical element 424 may comprise a resistor, e.g., electrical element 424 may comprise a cylindrical resistor. In one or more embodiments, electrical element 424 may comprise a radio frequency identification chip.

Illustratively, fixation mechanism 427 may be disposed in machine connector housing 420, e.g., fixation mechanism 427 may be disposed in machine connector housing 420 wherein a portion of fixation mechanism 427 contacts a portion of electrical element 424. In one or more embodiments, fixation mechanism 427 may be fixed in machine connector housing 420, e.g., fixation mechanism 427 may be fixed in machine connector housing 420 by an adhesive, an epoxy, a crimp, a weld, a friction fit, etc. Illustratively, fixation mechanism 427 may be configured to fix electrical element 424 in electrical element housing 435, e.g., fixation mechanism 427 may comprise a setscrew configured to fix electrical element 424 in electrical element housing 435. In one or more embodiments, fixation mechanism 427 may be electrically conductive. Illustratively, fixation mechanism 427 may be disposed in machine connector housing 420 wherein fixation mechanism 427 contacts electrical element 424 and electrical element 424 contacts machine connector 440, e.g., fixation mechanism 427 may be disposed in machine connector housing 420 wherein fixation mechanism 427 contacts electrical element superior end 426 and electrical element inferior end 425 contacts machine connector 440. In one or more embodiments, fixation mechanism 427 may be disposed in machine connector housing 420 wherein fixation mechanism 427 is electrically connected to electrical element 424 and electrical element 424 is electrically connected to machine connector 440.

Illustratively, machine coupler 450 may be configured to attach assembled optic fiber fixture 500 to a laser machine, e.g., machine interface 455 may be configured to attach assembled optic fiber fixture 500 to a laser machine. In one or more embodiments, machine interface 455 may comprise a magnet configured to attach assembled optic fiber fixture 500 to a laser machine. Illustratively, machine interface 455 may comprise an adhesive configured to attach assembled optic fiber fixture 500 to a laser machine. In one or more embodiments, assembled optic fiber fixture 500 may be reusable, e.g., assembled optic fiber fixture 500 may be sold non-sterile and not intended to be sterilized by a user in a medical autoclave. Illustratively, a user may clean assembled optic fiber fixture 500 by flushing assembled optic fiber fixture 500 with a syringe of isopropyl alcohol. In one or more embodiments, flushing assembled optic fiber fixture 500 with a syringe of isopropyl alcohol before each use of assembled optic fiber fixture 500 may be configured to remove any particulate matter that may have accumulated in assembled optic fiber fixture 500 since a previous use of assembled optic fiber fixture 500. Illustratively, optic fiber fixture 500 may comprise an end cap configured to fit over optic fiber fixture proximal end 502, e.g., optic fiber fixture 500 may comprise an end cap configured to fit over optic fiber fixture proximal end 502 to prevent particulate matter from accumulating in optic fiber fixture 500 when optic fiber fixture 500 is not being used by a user.

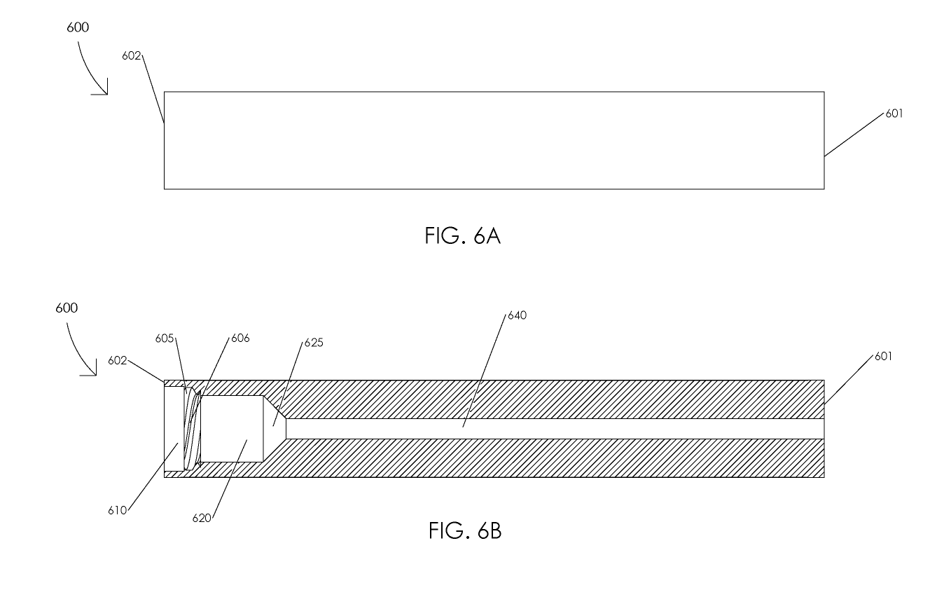

FIGS. 6A and 6B are schematic diagrams illustrating a handle facilitating sleeve 600. FIG. 6A illustrates a side view of a handle facilitating sleeve 600. FIG. 6B illustrates a cross-sectional view in a sagittal plane of a handle facilitating sleeve 600. In one or more embodiments, handle facilitating sleeve 600 may comprise a handle facilitating sleeve distal end 601 and a handle facilitating sleeve proximal end 602. Illustratively, handle facilitating sleeve 600 may be manufactured from a material configured to minimize a coefficient of friction between a portion of optic fiber 210 and a portion of handle facilitating sleeve 600, e.g., handle facilitating sleeve 600 may be manufactured from a self-lubricating thermoplastic material. In one or more embodiments, handle facilitating sleeve 600 may be manufactured from a material wherein a coefficient of friction between a portion of optic fiber 210 and a portion of handle facilitating sleeve 600 is in a range of 0.011 to 0.36, e.g., handle facilitating sleeve 600 may be manufactured from a material wherein a coefficient of friction between a portion of optic fiber 210 and a portion of handle facilitating sleeve 600 is 0.0311. Illustratively, handle facilitating sleeve 600 may be manufactured from a material wherein a coefficient of friction between a portion of optic fiber 210 and a portion of handle facilitating sleeve 600 is less than 0.011 or greater than 0.36. In one or more embodiments, handle facilitating sleeve 600 may be manufactured from a fluorocarbon material, e.g., handle facilitating sleeve 600 may be manufactured from a polytetrafluoroethylene material. Illustratively, handle facilitating sleeve 600 may be manufactured from an acetal-based polytetrafluoroethylene material, e.g., handle facilitating sleeve 600 may be manufactured from a turcite material. In one or more embodiments, handle facilitating sleeve 600 may be manufactured from a material having a density in a range of 0.024 to 0.073 pounds per cubic inch, e.g., handle facilitating sleeve 600 may be manufactured from a material having a density of 0.053 pounds per cubic inch. Illustratively, handle facilitating sleeve 600 may be manufactured from a material having a density of less than 0.024 pounds per cubic inch or greater than 0.073 pounds per cubic inch. In one or more embodiments, handle facilitating sleeve 600 may be manufactured from a material having a hardness in a range of 50 Shore D to 75 Shore D, e.g., handle facilitating sleeve 600 may be manufactured from a material having a hardness of 61 Shore D. Illustratively, handle facilitating sleeve 600 may be manufactured from a material having a hardness of less than 50 Shore D or greater than 75 Shore D. In one or more embodiments, optic fiber 210 may be manufactured from a material having a first hardness and handle facilitating sleeve 600 may be manufactured from a material having a second hardness. Illustratively, the first hardness may be greater than the second hardness. In one or more embodiments, optic fiber 210 may be manufactured from a material having a first hardness, handle facilitating sleeve 600 may be manufactured from a material having a second hardness, and transitory connector 100 may be manufactured from a material having a third hardness. Illustratively, the first hardness may be greater than the second hardness and the second hardness may be greater than the third hardness. In one or more embodiments, handle facilitating sleeve 600 may comprise a major diameter housing 605, a minor diameter housing 606, a second transitory connector housing 620, a second transitory connector housing proximal chamber 610, a second transitory connector housing distal taper 625, and a handle facilitating sleeve inner bore 640. Illustratively, second transitory connector housing distal taper 625 may be disposed between handle facilitating sleeve inner bore 640 and second transitory connector housing 620. In one or more embodiments, major diameter housing 605 may be disposed between second transitory connector housing proximal chamber 610 and second transitory connector housing 620. Illustratively, minor diameter housing 606 may be disposed between second transitory connector housing proximal chamber 610 and second transitory connector housing 620.

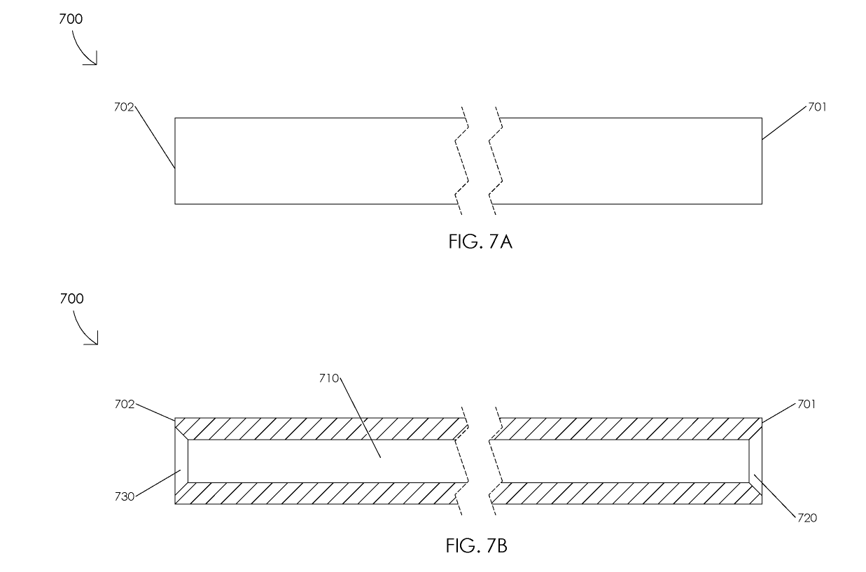

FIGS. 7A and 7B are schematic diagrams illustrating a hypodermic tube 700. FIG. 7A illustrates a side view of a hypodermic tube 700. FIG. 7B illustrates a cross-sectional view in a sagittal plane of a hypodermic tube 700. Illustratively, hypodermic tube 700 may comprise a hypodermic tube distal end 701 and a hypodermic tube proximal end 702. Illustratively, hypodermic tube 700 may comprise a hypodermic tube through lumen 710, a hypodermic tube distal taper 720, and a hypodermic tube proximal taper 730. Illustratively, hypodermic tube 700 may be manufactured from any suitable material, e.g., polymers, metals, metal alloys, etc., or from any combination of suitable materials.

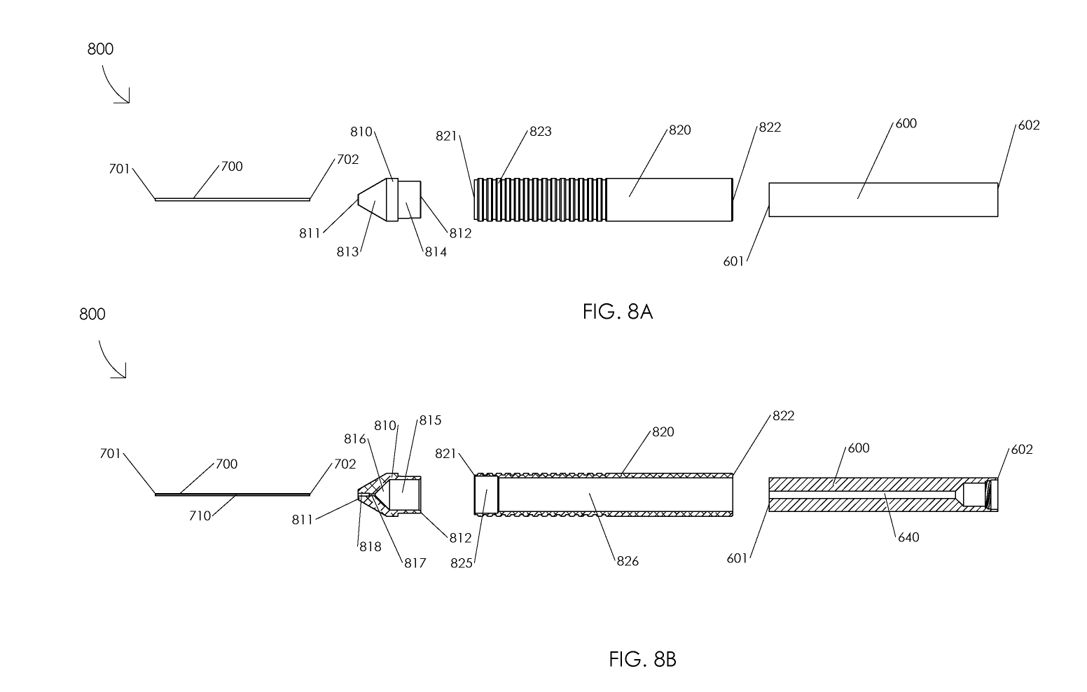

FIGS. 8A and 8B are schematic diagrams illustrating an exploded view of a reusable handle assembly 800. FIG. 8A illustrates a side view of a reusable handle assembly 800. FIG. 8B illustrates a cross-sectional view in a sagittal plane of a reusable handle assembly 800. In one or more embodiments, a reusable handle assembly 800 may comprise a hypodermic tube 700, a handle nosecone 810, a handle base 820, and a handle facilitating sleeve 600. Illustratively, handle nosecone 810 may comprise a handle nosecone distal end 811 and a handle nosecone proximal end 812. In one or more embodiments, handle nosecone 810 may comprise a handle nosecone tapered portion 813 and a handle nosecone base 814. Illustratively, handle nosecone 810 may comprise a handle nosecone inner bore 815, a handle nosecone distal taper 816, an optic fiber distal end guide 817, and a hypodermic tube housing 818. In one or more embodiments, handle base 820 may comprise a handle base distal end 821 and a handle base proximal end 822. Illustratively, handle base 820 may comprise a grip portion 823. In one or more embodiments, handle base 820 may comprise a nosecone housing 825 and a handle facilitating sleeve housing 826.

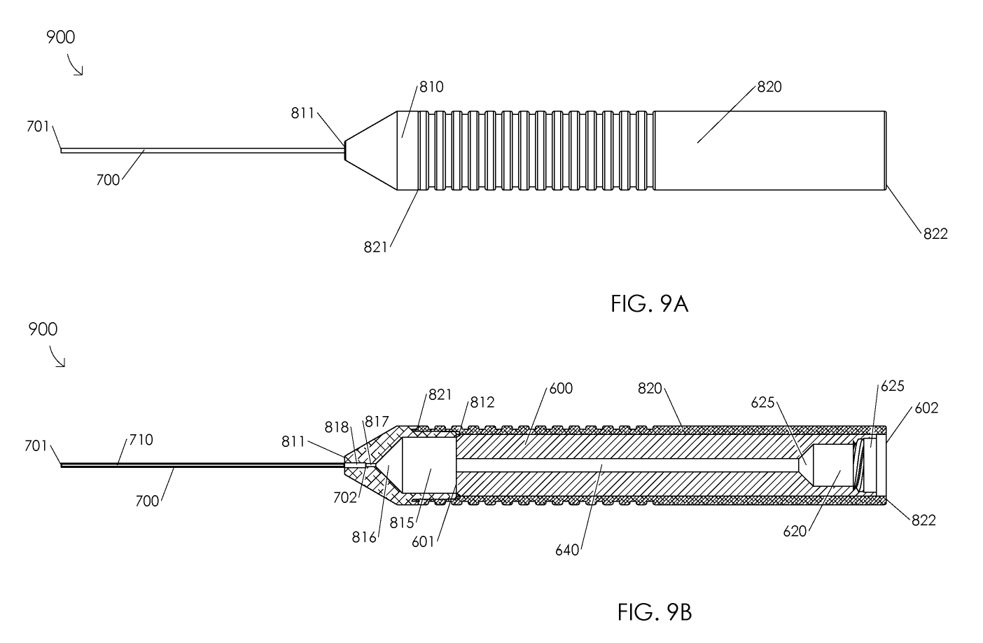

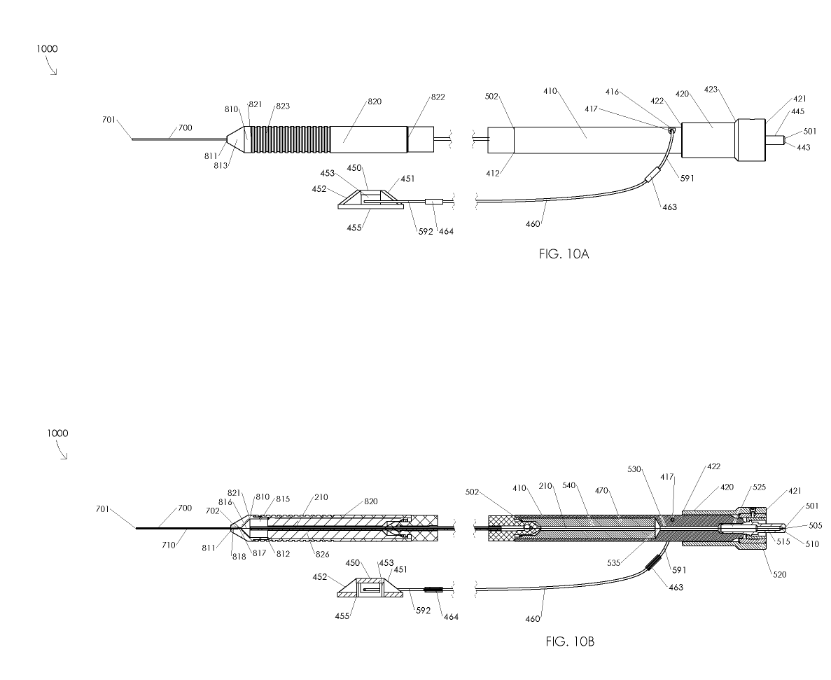

FIGS. 9A and 9B are schematic diagrams illustrating an assembled reusable handle 900. FIG. 9A illustrates a side view of an assembled reusable handle 900. FIG. 9B illustrates a cross-sectional view in a sagittal plane of an assembled reusable handle 900. Illustratively, handle facilitating sleeve 600 may be disposed in a portion of handle base 820, e.g., handle facilitating sleeve 600 may be disposed in handle facilitating sleeve housing 826. In one or more embodiments, handle facilitating sleeve 600 may be disposed in a portion of handle base 820 wherein handle facilitating sleeve proximal end 602 is adjacent to handle base proximal end 822, e.g., handle facilitating sleeve 600 may be disposed in a portion of handle base 820 wherein handle facilitating sleeve proximal end 602 abuts handle base proximal end 822. Illustratively, handle facilitating sleeve 600 may be fixed in a portion of handle base 820, e.g., handle facilitating sleeve 600 may be fixed in a portion of handle base 820 by an adhesive, an epoxy, a crimp, a weld, a friction fit, etc. In one or more embodiments, handle facilitating sleeve 600 may be fixed in handle facilitating sleeve housing 826, e.g., handle facilitating sleeve 600 may be fixed in handle facilitating sleeve housing 826 by an adhesive, an epoxy, a crimp, a weld, a friction fit, etc.

Illustratively, a portion of handle nosecone 810 may be disposed in a portion of handle base 820, e.g., handle nosecone proximal end 812 may be disposed in nosecone housing 825. In one or more embodiments, handle nosecone base 814 may be disposed in a portion of handle base 820, e.g., handle nosecone base 814 may be disposed in a portion of handle base 820 wherein handle nosecone distal end 811 extends out from handle base distal end 821. Illustratively, a portion of handle nosecone 810 may be disposed in a portion of handle base 820 wherein handle nosecone proximal end 812 is adjacent to handle facilitating sleeve distal end 601, e.g., a portion of handle nosecone 810 may be disposed in a portion of handle base 820 wherein handle nosecone proximal end 812 abuts handle facilitating sleeve distal end 601. In one or more embodiments, a portion of handle nosecone 810 may be fixed in a portion of handle base 820, e.g., a portion of handle nosecone 810 may be fixed in a portion of handle base 820 by an adhesive, an epoxy, a crimp, a weld, a friction fit, etc. Illustratively, a portion of handle nosecone 810 may be fixed in handle nosecone housing 825, e.g., a portion of handle nosecone 810 may be fixed in handle nosecone housing 825 by an adhesive, an epoxy, a crimp, a weld, a friction fit, etc.

In one or more embodiments, a portion of hypodermic tube 700 may be disposed in a portion of handle nosecone 810, e.g., hypodermic tube proximal end 702 may be disposed in hypodermic tube housing 818. Illustratively, a portion of hypodermic tube 700 may be disposed in a portion of handle nosecone 810 wherein hypodermic tube distal end 701 extends out from handle nosecone distal end 811. In one or more embodiments, a portion of hypodermic tube 700 may be fixed in a portion of handle nosecone 810, e.g., a portion of hypodermic tube 700 may be fixed in a portion of handle nosecone 810 by an adhesive, an epoxy, a crimp, a weld, a friction fit, etc. Illustratively, a portion of hypodermic tube 700 may be fixed in hypodermic tube housing 818, e.g., a portion of hypodermic tube 700 may be fixed in hypodermic tube housing 818 by an adhesive, an epoxy, a crimp, a weld, a friction fit, etc.

FIGS. 10A and 10B are schematic diagrams illustrating an assembled reusable laser probe with single-use optic fiber 1000. FIG. 10A illustrates a side view of an assembled reusable laser probe with single-use optic fiber 1000. FIG. 10B illustrates a cross-sectional view in a sagittal plane of an assembled reusable laser probe with single-use optic fiber 1000. Illustratively, an assembled reusable laser probe with single-use optic fiber 1000 may comprise an assembled single-use optic fiber 300, an assembled optic fiber fixture 500, and an assembled reusable handle 900. In one or more embodiments, an assembled reusable laser probe with single-use optic fiber 1000 may comprise a first transitory connector 100 disposed in a portion of assembled optic fiber fixture 500 and a second transitory connector 100 disposed in apportion of assembled reusable handle 900.

In one or more embodiments, an assembled reusable laser probe with single-use optic fiber 1000 may comprise a first transitory connector 100 disposed in a portion of assembled optic fiber fixture 500 wherein optic fiber proximal end 212 is adjacent to assembled optic fiber fixture distal end 501. Illustratively, an assembled reusable laser probe with single-use optic fiber 1000 may comprise a first transitory connector 100 disposed in a portion of assembled optic fiber fixture 500 wherein optic fiber proximal end 212 abuts assembled optic fiber fixture distal end 501. In one or more embodiments, an assembled reusable laser probe with single-use optic fiber 1000 may comprise a first transitory connector 100 disposed in a portion of assembled optic fiber fixture 500 wherein optic fiber proximal end 212 is coplanar with assembled optic fiber fixture distal end 501. Illustratively, inserting a portion of optic fiber 210 into a portion of assembled optic fiber fixture 500 may be configured to ingress optic fiber proximal end 212 into first transitory connector housing 550. In one or more embodiments, inserting a portion of optic fiber 210 into a portion of assembled optic fiber fixture 500 may be configured to ingress optic fiber proximal end 212 into first transitory connector housing distal taper 545. Illustratively, first transitory connector housing distal taper 545 may be configured to guide an ingress of optic fiber 210 into fixture facilitating sleeve inner bore 540. In one or more embodiments, inserting a portion of optic fiber 210 into a portion of assembled optic fiber fixture 500 may be configured to ingress optic fiber proximal end 212 into fixture facilitating sleeve inner bore 540. Illustratively, inserting a portion of optic fiber 210 into a portion of assembled optic fiber fixture 500 may be configured to ingress optic fiber proximal end 212 into fixture facilitating sleeve housing 535. In one or more embodiments, fixture facilitating sleeve housing 535 may be configured to guide an ingress of optic fiber 210 into fixture base inner bore 530. Illustratively, inserting a portion of optic fiber 210 into a portion of assembled optic fiber fixture 500 may be configured to ingress optic fiber proximal end 212 into fixture base inner bore 530. In one or more embodiments, inserting a portion of optic fiber 210 into a portion of assembled optic fiber fixture 500 may be configured to ingress optic fiber proximal end 212 into machine connector proximal taper 444. Illustratively, machine connector proximal taper 444 may be configured to guide an ingress of optic fiber 210 into machine connector proximal inner lumen 525. In one or more embodiments, inserting a portion of optic fiber 210 into a portion of assembled optic fiber fixture 500 may be configured to ingress optic fiber proximal end 212 into machine connector proximal inner lumen 525. Illustratively, inserting a portion of optic fiber 210 into a portion of assembled optic fiber fixture 500 may be configured to ingress optic fiber proximal end 212 into inner lumen proximal taper 520. In one or more embodiments, inner lumen proximal taper 520 may be configured to guide an ingress of optic fiber into machine connector distal inner lumen 515. Illustratively, inserting a portion of optic fiber 210 into a portion of assembled optic fiber fixture 500 may be configured to ingress optic fiber proximal end 212 into machine connector distal inner lumen 515. In one or more embodiments, inserting a portion of optic fiber 210 into a portion of assembled optic fiber fixture 500 may be configured to ingress optic fiber proximal end 212 into inner lumen distal taper 510. Illustratively, inner lumen distal taper 510 may be configured to guide an ingress of optic fiber 210 into optic fiber proximal end guide 505. In one or more embodiments, inserting a portion of optic fiber 210 into a portion of assembled optic fiber fixture 500 may be configured to ingress optic fiber proximal end 212 into optic fiber proximal end guide 505.

Illustratively, inserting first transitory connector 100 into a portion of assembled optic fiber fixture 500 may be configured to ingress optic fiber 210 into optic fiber proximal end guide 505, e.g., inserting first transitory connector 100 into a portion of assembled optic fiber fixture 500 may be configured to ingress optic fiber 210 into optic fiber proximal end guide 505 wherein optic fiber proximal end 212 is coplanar with assembled optic fiber fixture distal end 501. In one or more embodiments, inserting first transitory connector distal end 101 into first transitory connector housing distal taper 545 may be configured to ingress optic fiber 210 into optic fiber proximal end guide 505, e.g., inserting first transitory connector distal end 101 into first transitory connector housing distal taper 545 may be configured to ingress optic fiber 210 into optic fiber proximal end guide 505 wherein optic fiber proximal end 212 is coplanar with assembled optic fiber fixture distal end 501. Illustratively, inserting first transitory connector nosecone base 120 into first transitory connector housing 550 may be configured to ingress optic fiber 210 into optic fiber proximal end guide 505, e.g., inserting first transitory connector nosecone base 120 into first transitory connector housing 550 may be configured to ingress optic fiber 210 into optic fiber proximal end guide 505 wherein optic fiber proximal end 212 is coplanar with assembled optic fiber fixture distal end 501. In one or more embodiments, inserting major diameter 105 and minor diameter 106 into first transitory connector housing threading 560 may be configured to ingress optic fiber 210 into optic fiber proximal end guide 505, e.g., inserting major diameter 105 and minor diameter 106 into first transitory connector housing threading 560 may be configured to ingress optic fiber 210 into optic fiber proximal end guide 505 wherein optic fiber proximal end 212 is coplanar with assembled optic fiber fixture distal end 501. Illustratively, rotating major diameter 105 and minor diameter 106 in first transitory connector housing threading 560 may be configured to ingress optic fiber 210 into optic fiber proximal end guide 505, e.g., rotating major diameter 105 and minor diameter 106 in first transitory connector housing threading 560 may be configured to ingress optic fiber 210 into optic fiber proximal end guide 505 wherein optic fiber proximal end 212 is coplanar with assembled optic fiber fixture distal end 501. In one or more embodiments, rotating major diameter 105 and minor diameter 106 in first transitory connector housing threading 560 may be configured to fix first transitory connector 100 in assembled optic fiber fixture 500, e.g., rotating major diameter 105 and minor diameter 106 in first transitory connector housing threading 560 may be configured to fix major diameter 105 and minor diameter 106 in first transitory connector housing threading 560. For example, major diameter 105 and minor diameter 106 may comprise a threaded fastener.

Illustratively, inserting a portion of optic fiber 210 into a portion of assembled reusable handle 900 may be configured to ingress optic fiber distal end 211 into second transitory connector housing 620. In one or more embodiments, inserting a portion of optic fiber 210 into a portion of assembled reusable handle 900 may be configured to ingress optic fiber distal end 211 into second transitory connector housing distal taper 625. Illustratively, second transitory connector housing distal taper 625 may be configured to guide an ingress of optic fiber 210 into handle facilitating sleeve inner bore 640. In one or more embodiments, inserting a portion of optic fiber 210 into a portion of assembled reusable handle 900 may be configured to ingress optic fiber distal end 211 into handle facilitating sleeve inner bore 640. Illustratively, inserting a portion of optic fiber 210 into a portion of assembled reusable handle 900 may be configured to ingress optic fiber distal end 211 into handle nosecone inner bore 815. In one or more embodiments, inserting a portion of optic fiber 210 into a portion of assembled reusable handle 900 may be configured to ingress optic fiber distal end 211 into handle nosecone distal taper 816. Illustratively, handle nosecone distal taper 816 may be configured to guide an ingress of optic fiber 210 into optic fiber distal end guide 817. In one or more embodiments, inserting a portion of optic fiber 210 into a portion of assembled reusable handle 900 may be configured to ingress optic fiber distal end 211 into optic fiber distal end guide 817. Illustratively, inserting a portion of optic fiber 210 into a portion of assembled reusable handle 900 may be configured to ingress optic fiber distal end 211 into hypodermic tube proximal taper 730. In one or more embodiments, hypodermic tube proximal taper 730 may be configured to guide an ingress of optic fiber into hypodermic tube through lumen 710. Illustratively, inserting a portion of optic fiber 210 into a portion of assembled reusable handle 900 may be configured to ingress optic fiber distal end 211 into hypodermic tube through lumen 710.

Illustratively, inserting second transitory connector 100 into a portion of assembled reusable handle 900 may be configured to ingress optic fiber 210 into hypodermic tube through lumen 710, e.g., inserting second transitory connector 100 into a portion of assembled reusable handle 900 may be configured to ingress optic fiber 210 into hypodermic tube through lumen 710 wherein optic fiber distal end 211 is adjacent to hypodermic tube distal end 701. In one or more embodiments, inserting second transitory connector 100 into a portion of assembled reusable handle 900 may be configured to ingress optic fiber 210 into hypodermic tube through lumen 710 wherein optic fiber distal end 211 is coplanar with hypodermic tube distal end 701. Illustratively, inserting second transitory connector distal end 101 into second transitory connector housing distal taper 625 may be configured to ingress optic fiber 210 into hypodermic tube through lumen 710, e.g., inserting second transitory connector distal end 101 into second transitory connector housing distal taper 625 may be configured to ingress optic fiber 210 into hypodermic tube through lumen 710 wherein optic fiber distal end 211 is adjacent to hypodermic tube distal end 701. In one or more embodiments, inserting second transitory connector distal end 101 into second transitory connector housing distal taper 625 may be configured to ingress optic fiber 210 into hypodermic tube through lumen 710 wherein optic fiber distal end 211 is coplanar with hypodermic tube distal end 701. Illustratively, inserting second transitory connector nosecone base 120 into second transitory connector housing 620 may be configured to ingress optic fiber 210 into hypodermic tube through lumen 710, e.g., inserting second transitory connector nosecone base 120 into second transitory connector housing 620 may be configured to ingress optic fiber 210 into hypodermic tube through lumen 710 wherein optic fiber distal end 211 is adjacent to hypodermic tube distal end 701. In one or more embodiments, inserting second transitory connector nosecone base 120 into second transitory connector housing 620 may be configured to ingress optic fiber 210 into hypodermic tube through lumen 710 wherein optic fiber distal end 211 is coplanar with hypodermic tube distal end 701. Illustratively, inserting major diameter 105 in major diameter housing 605 may be configured to ingress optic fiber 210 into hypodermic tube through lumen 710, e.g., inserting major diameter 105 in major diameter housing 605 may be configured to ingress optic fiber 210 into hypodermic tube through lumen 710 wherein optic fiber distal end 211 is adjacent to hypodermic tube distal end 701. In one or more embodiments, inserting major diameter 105 in major diameter housing 605 may be configured to ingress optic fiber 210 into hypodermic tube through lumen 710 wherein optic fiber distal end 211 is coplanar with hypodermic tube distal end 701. Illustratively, inserting minor diameter 106 into minor diameter housing 606 may be configured to ingress optic fiber 210 into hypodermic tube through lumen 710, e.g., inserting minor diameter 106 into minor diameter housing 606 may be configured to ingress optic fiber 210 into hypodermic tube through lumen 710 wherein optic fiber distal end 211 is adjacent to hypodermic tube distal end 701. In one or more embodiments, inserting minor diameter 106 into minor diameter housing 606 may be configured to ingress optic fiber 210 into hypodermic tube through lumen 710 wherein optic fiber distal end 211 is coplanar with hypodermic tube distal end 701. Illustratively, rotating major diameter 105 in major diameter housing 605 may be configured to ingress optic fiber 210 into hypodermic tube through lumen 710, e.g., rotating major diameter 105 in major diameter housing 605 may be configured to ingress optic fiber 210 into hypodermic tube through lumen 710 wherein optic fiber distal end 211 is adjacent to hypodermic tube distal end 701. In one or more embodiments, rotating major diameter 105 in major diameter housing 605 may be configured to ingress optic fiber 210 into hypodermic tube through lumen 710 wherein optic fiber distal end 211 is coplanar with hypodermic tube distal end 701. Illustratively, rotating minor diameter 106 in minor diameter housing 606 may be configured to ingress optic fiber 210 into hypodermic tube through lumen 710, e.g., rotating minor diameter 106 in minor diameter housing 606 may be configured to ingress optic fiber 210 into hypodermic tube through lumen 710 wherein is optic fiber distal end 211 is adjacent to hypodermic tube distal end 701. In one or more embodiments, rotating minor diameter 106 in minor diameter housing 606 may be configured to ingress optic fiber 210 into hypodermic tube through lumen 710 wherein optic fiber distal end 211 is coplanar with hypodermic tube distal end 701. Illustratively, rotating major diameter 105 in major diameter housing 605 may be configured to fix second transitory connector 100 in assembled reusable handle 900, e.g., rotating major diameter 105 in major diameter housing 605 may be configured to fix major diameter 105 in major diameter housing 605. In one or more embodiments, rotating minor diameter 106 in minor diameter housing 606 may be configured to fix second transitory connector 100 in assembled reusable handle 900, e.g., rotating minor diameter 106 in minor diameter housing 606 may be configured to fix minor diameter 106 in minor diameter housing 606. For example, major diameter 105 and minor diameter 106 may comprise a threaded fastener.

Illustratively, hypodermic tube distal taper 720 may be configured to prevent degradation to a laser spot wherein optic fiber distal end 211 is recessed from hypodermic tube distal end 701 relative to a laser spot wherein optic fiber distal end 211 is coplanar with hypodermic tube distal end 701. In one or more embodiments, hypodermic tube distal taper 720 may be configured to ensure that a power output wherein optic fiber distal end 211 is recessed relative to hypodermic tube distal end 701 is identical to a power output wherein optic fiber distal end 211 is coplanar with hypodermic tube distal end 701. Illustratively, hypodermic tube distal taper 720 may be configured to ensure that a laser spot size wherein optic fiber distal end 211 is recessed relative to hypodermic tube distal end 701 is identical to a laser spot size wherein optic fiber distal end 211 is coplanar with hypodermic tube distal end 701. In one or more embodiments, hypodermic tube distal taper 720 may be configured to ensure that a laser spot shape wherein optic fiber distal end 211 is recessed relative to hypodermic tube distal end 701 is identical to a laser spot shape wherein optic fiber distal end 211 is coplanar with hypodermic tube distal end 701.

Illustratively, a user may perform a photocoagulation procedure with an assembled reusable laser probe with single-use optic fiber 1000. In one or more embodiments, a user may connect machine connector 440 to a laser machine. Illustratively, a user may energize the laser machine to deliver laser light into optic fiber proximal end 212, through optic fiber 210, out from optic fiber distal end 211, and onto a surgical target site. In one or more embodiments, assembled reusable handle 900 may be a reusable medical device sold non-sterile and sterilized by a user in a medical autoclave.

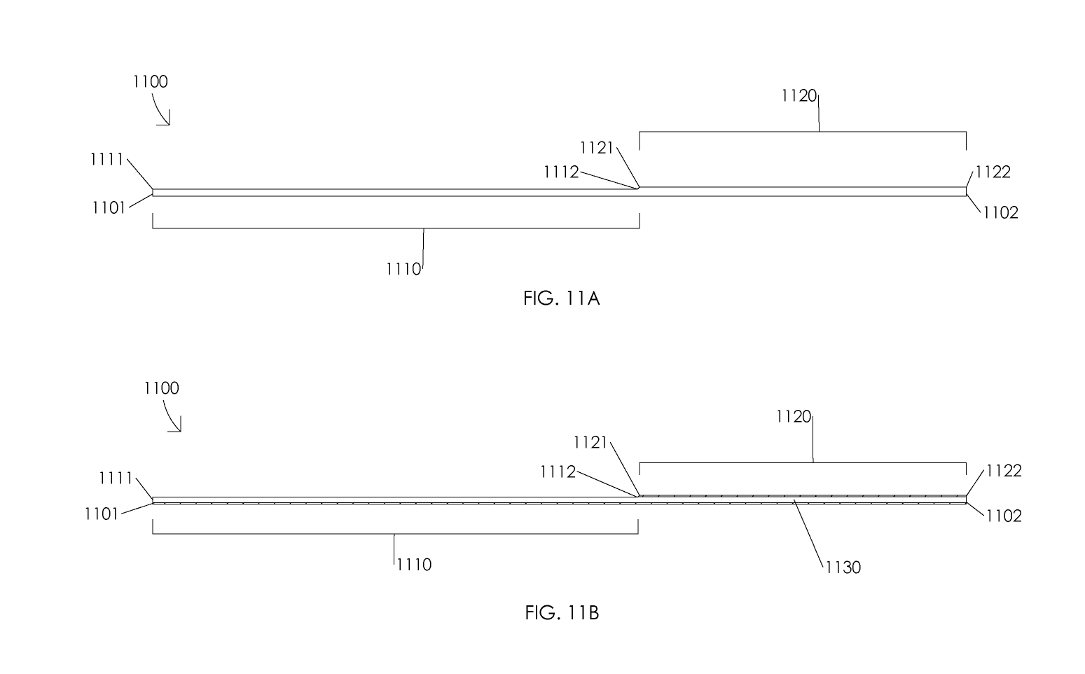

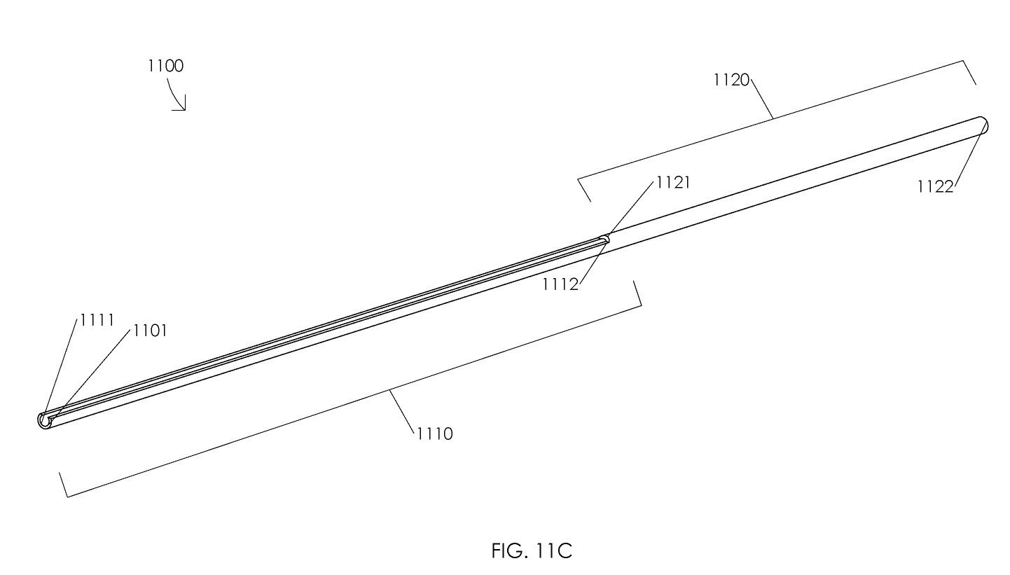

FIGS. 11A, 11B, and 11C are schematic diagrams illustrating an optic fiber support mechanism 1100. FIG. 11A illustrates a side view of an optic fiber support mechanism 1100. FIG. 11B illustrates a cross-sectional view in a sagittal plane of an optic fiber support mechanism 1100. FIG. 11C illustrates an isometric view of an optic fiber support mechanism 1100. Illustratively, an optic fiber support mechanism 1100 may comprise an optic fiber support mechanism distal end 1101 and an optic fiber support mechanism proximal end 1102. In one more embodiments, optic fiber support mechanism 1100 may comprise an anterior portion 1110 having an anterior portion distal end 1111 and an anterior portion proximal end 1112. Illustratively, anterior portion 1110 may comprise a channel or an aperture extending between anterior portion distal end 1111 and anterior portion proximal end 1112. In one or more embodiments, optic fiber support mechanism 1100 may comprise a posterior portion 1120 having a posterior portion distal end 1121 and a posterior portion proximal end 1122. Illustratively, optic fiber support mechanism 1100 may comprise an optic fiber support mechanism guide 1130.

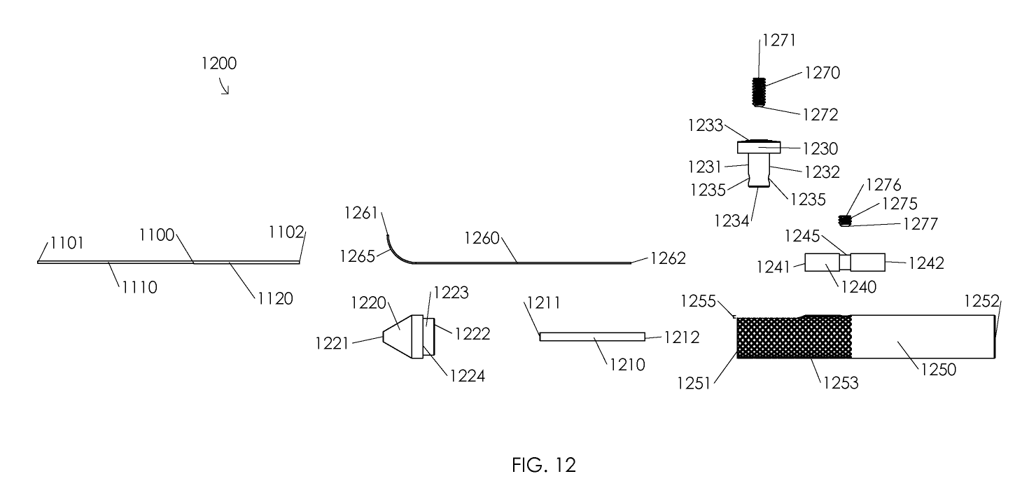

FIG. 12 is a schematic diagram illustrating a reusable steerable handle assembly 1200. Illustratively, a reusable steerable handle assembly 1200 may comprise an optic fiber support mechanism 1100, a piston tube 1210, a steerable nosecone 1220, a control mechanism 1230, a piston tube proximal housing 1240, a steerable handle base 1250, a housing tube 1260, a distal fixation mechanism 1270, and a proximal fixation mechanism 1275. In one or more embodiments, piston tube 1210 may comprise a piston tube distal end 1211 and a piston tube proximal end 1212. Illustratively, steerable nosecone 1220 may comprise a steerable nosecone distal end 1221 and a steerable nosecone proximal end 1222. In one or more embodiments, steerable nosecone 1220 may comprise a steerable nosecone base 1223 and a steerable handle base interface 1224. Illustratively, control mechanism 1230 may comprise a control mechanism distal end 1231, a control mechanism proximal end 1232, a control mechanism superior end 1233, and a control mechanism inferior end 1234. In one or more embodiments, control mechanism 1230 may comprise a control mechanism inner bore 1235. Illustratively, piston tube proximal housing 1240 may comprise a piston tube proximal housing distal end 1241 and a piston tube proximal housing proximal end 1242. In one or more embodiments, piston tube proximal housing 1240 may comprise a proximal fixation mechanism housing 1245. Illustratively, steerable handle base 1250 may comprise a steerable handle base distal end 1251 and a steerable handle base proximal end 1252. In one or more embodiments, steerable handle base 1250 may comprise a knurled portion 1253 and control mechanism guide 1255. Illustratively, housing tube 1260 may comprise a housing tube distal end 1261 and a housing tube proximal end 1262. In one or more embodiments, housing tube 1260 may comprise a curved portion 1265. Illustratively, distal fixation mechanism 1270 may comprise a distal fixation mechanism superior end 1271 and a distal fixation mechanism inferior end 1272. In one or more embodiments, proximal fixation mechanism 1275 may comprise a proximal fixation mechanism superior end 1276 and a proximal fixation mechanism inferior end 1277.

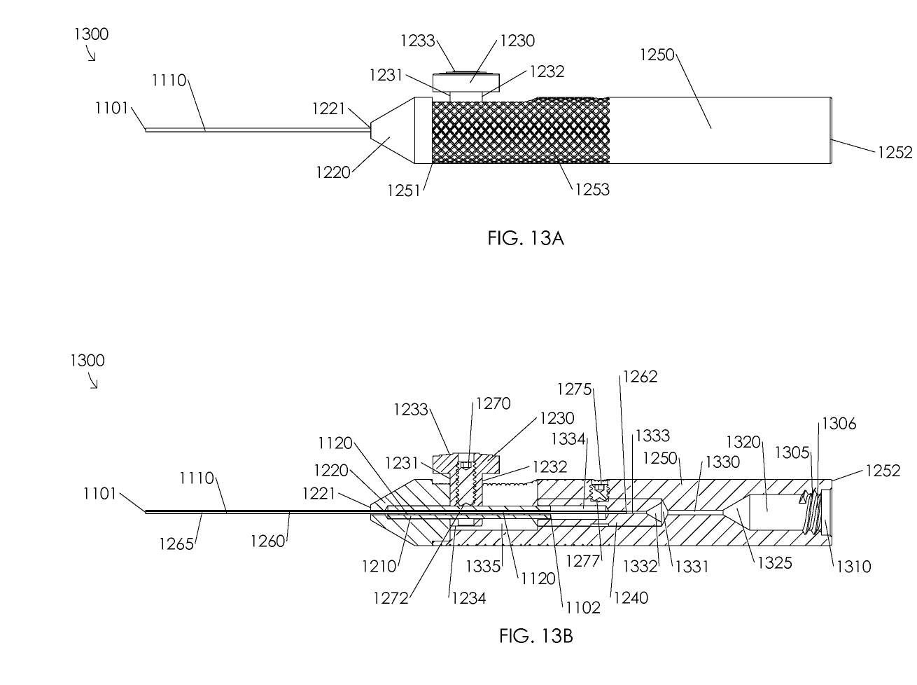

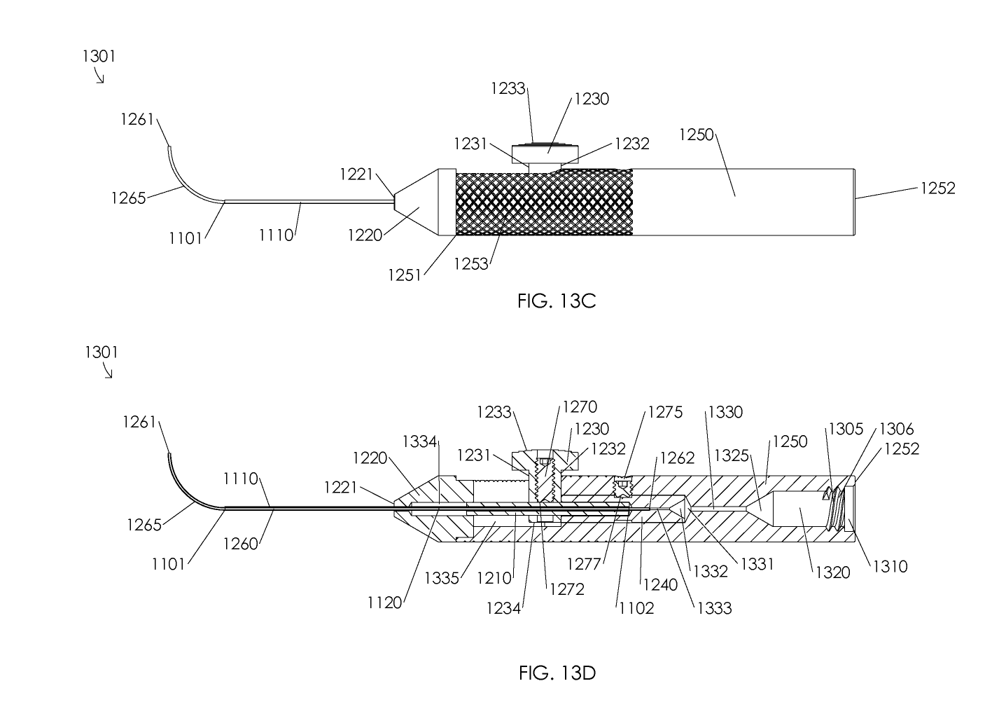

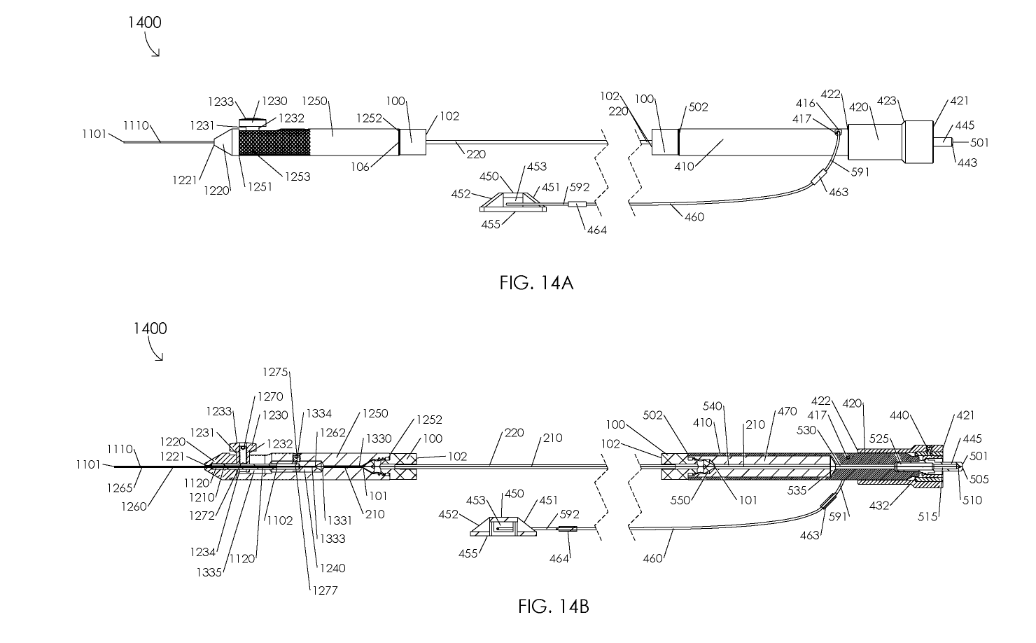

FIGS. 13A, 13B, 13C, and 13D are schematic diagrams illustrating an assembled reusable steerable handle. FIG. 13A illustrates a side view of an assembled reusable steerable handle with straight housing tube 1300. FIG. 13B illustrates a cross-sectional view in a sagittal plane of an assembled reusable steerable handle with straight housing tube 1300. FIG. 13C illustrates a side view of an assembled reusable steerable handle with curved housing tube 1301. FIG. 13D illustrates a cross-sectional view in a sagittal plane of an assembled reusable steerable handle with curved housing tube 1301. Illustratively, an assembled reusable steerable handle may comprise a major diameter housing 1305, a minor diameter housing 1306, a second transitory connector housing 1320, a second transitory connector housing proximal chamber 1310, a second transitory connector housing distal taper 1325, a steerable handle inner bore 1330, a steerable handle inverted taper 1331, a steerable handle inner taper 1332, a housing tube housing 1333, a piston tube guide 1334, and a control mechanism housing 1335.

In one or more embodiments, piston tube 1210 may be disposed in steerable nosecone 1220, steerable handle base 1250, control mechanism inner bore 1235, piston tube proximal housing 1240, piston tube guide 1334, and control mechanism housing 1335. Illustratively, piston tube 1210 may be disposed in control mechanism inner bore 1235 wherein piston tube distal end 1211 extends out from control mechanism distal end 1231. In one or more embodiments, piston tube 1210 may be disposed in control mechanism inner bore 1235 wherein piston tube proximal end 1212 extend out from control mechanism proximal end 1232. Illustratively, piston tube 1210 may be fixed in control mechanism inner bore 1235, e.g., piston tube 1210 may be fixed in control mechanism inner bore 1235 by an adhesive, an epoxy, a crimp, a weld, a friction fit, a setscrew, etc. In one or more embodiments, distal fixation mechanism 1270 may be configured to fix piston tube 1210 in control mechanism inner bore 1235, e.g., distal fixation mechanism 1270 may be disposed in a portion of control mechanism wherein distal fixation mechanism inferior end 1272 abuts a portion of piston tube 1210. Illustratively, distal fixation mechanism 1270 may apply a force to a portion of piston tube 1210 configured to fix piston tube 1210 in control mechanism inner bore 1235.

In one or more embodiments, a portion of steerable nosecone 1220 may be disposed in a portion of steerable handle base 1250, e.g., steerable nosecone proximal end 1222 may be disposed in a portion of steerable handle base 1250. Illustratively, a portion of steerable nosecone 1220 may be disposed in a portion of steerable handle base 1250 wherein steerable handle base interface 1224 is adjacent to steerable handle base distal end 1251, e.g., a portion of steerable nosecone 1220 may be disposed in a portion of steerable handle base 1250 wherein steerable handle base interface 1224 abuts steerable handle base distal end 1251. Illustratively, steerable nosecone base 1223 may be disposed in a portion of steerable handle base 1250, e.g., steerable nosecone base 1223 may be disposed in a portion of steerable handle base 1250 wherein steerable nosecone distal end 1221 extends out from steerable handle base distal end 1251. In one or more embodiments, a portion of steerable nosecone 1220 may be fixed in a portion of steerable handle base 1250, e.g., a portion of steerable nosecone 1220 may be fixed in a portion of steerable handle base 1250 by an adhesive, an epoxy, a crimp, a weld, a friction fit, a setscrew, etc.

In one or more embodiments, piston tube 1210 may be disposed in steerable nosecone 1220 wherein piston tube distal end 1211 is disposed in steerable nosecone 1220. Illustratively, piston tube 1210 may be disposed in piston tube proximal housing 1240 wherein piston tube proximal end 1212 is disposed in piston tube proximal housing 1240. In one or more embodiments, piston tube 1220 may be disposed in piston tube guide 1334 wherein piston tube 1220 is configured to actuate within piston tube guide 1334. Illustratively, control mechanism 1230 may be disposed in control mechanism housing 1335 and control mechanism guide 1255. In one or more embodiments, control mechanism 1230 may be disposed in control mechanism housing 1335 wherein a portion of control mechanism 1230 extends out from control mechanism housing 1335, e.g., control mechanism 1230 may be disposed in control mechanism housing 1335 wherein control mechanism superior end 1233 extends out from control mechanism housing 1335. Illustratively, control mechanism 1230 may be configured to actuate within control mechanism guide 1255. In one or more embodiments, control mechanism 1230 may be configured to actuate within control mechanism housing 1335.

Illustratively, piston tube proximal housing 1240 may be disposed in an inner portion of steerable handle base 1250, e.g., piston tube proximal housing 1240 may be disposed in an inner portion of handle base 1250 wherein piston tube proximal housing distal end 1241 is disposed in the inner portion of steerable handle base 1250 and wherein piston tube proximal housing proximal end 1242 is disposed in the inner portion of steerable handle base 1250. In one or more embodiments, piston tube proximal housing 1240 may be fixed in an inner portion of steerable handle base 1250, e.g., piston tube proximal housing 1240 may be fixed in an inner portion of steerable handle base 1250 by an adhesive, an epoxy, a crimp, a weld, a friction fit, a setscrew, etc. Illustratively, proximal fixation mechanism 1275 may be configured to fix piston tube proximal housing 1240 in an inner portion of steerable handle base 1250, e.g., proximal fixation mechanism 1275 may be disposed in a portion of steerable handle base 1250 wherein proximal fixation mechanism inferior end 1277 abuts a portion of piston tube proximal housing 1240. In one or more embodiments, proximal fixation mechanism 1275 may apply a force to a portion of piston tube proximal housing 1240 configured to fix piston tube proximal housing 1240 in an inner portion of steerable handle base 1250.

Illustratively, optic fiber support mechanism 1100 may be disposed in steerable nosecone 1220, steerable handle base 1250, piston tube 1210, piston tube guide 1334, control mechanism inner bore 1235, control mechanism housing 1335, and piston tube proximal housing 1240. In one or more embodiments, optic fiber support mechanism 1100 may be disposed in steerable handle base 1250 wherein posterior portion 1120 is completely disposed in steerable handle base 1250, e.g., optic fiber support mechanism 1100 may be disposed in steerable handle base 1250 wherein posterior portion distal end 1121 is disposed between steerable handle base distal end 1251 and steerable handle base proximal end 1252 and wherein poster portion proximal end 1122 is disposed between steerable handle base distal end 1251 and steerable handle base proximal end 1252. Illustratively, optic fiber support mechanism 1100 may be disposed in steerable nosecone 1220 wherein optic fiber support mechanism distal end 1101 extends out from steerable nosecone distal end 1221, e.g., optic fiber support mechanism 1100 may be disposed in steerable nosecone 1220 wherein anterior portion 1110 extends out from steerable nosecone distal end 1221. In one or more embodiments, optic fiber support mechanism 1100 may be disposed in piston tube 1210 wherein optic fiber support mechanism proximal end 1102 is adjacent to piston tube proximal end 1212, e.g., optic fiber support mechanism 1100 may be disposed in piston tube 1210 wherein optic fiber support mechanism proximal end 1102 is coplanar with piston tube proximal end 1212. Illustratively, optic fiber support mechanism 1100 may be fixed in piston tube 1210, e.g., optic fiber support mechanism 1100 may be fixed in piston tube 1210 by an adhesive, an epoxy, a crimp, a weld, a friction fit, a setscrew, etc. In one or more embodiments, optic fiber support mechanism guide 1100 may be configured to actuate within piston tube guide 1334. Illustratively, an actuation of piston tube 1210 within piston tube guide 1334 may be configured to actuate optic fiber support mechanism 1100 within piston tube guide 1334.

In one or more embodiments, housing tube 1260 may be disposed in optic fiber support mechanism 1100, optic fiber support mechanism guide 1130, steerable nosecone 1220, steerable handle base 1250, piston tube 1210, piston tube guide 1334, control mechanism inner bore 1235, control mechanism housing 1335, piston tube proximal housing 1240, and housing tube housing 1333. Illustratively, housing tube 1260 may be disposed in steerable handle base 1250 wherein housing tube distal end 1261 extends out from steerable nosecone distal end 1221. In one or more embodiments, housing tube 1260 may be disposed in piston tube proximal housing 1240 wherein housing tube proximal end 1262 is disposed in housing tube housing 1333. Illustratively, housing tube 1260 may be fixed in housing tube housing 1333, e.g., housing tube 1260 may be fixed in housing tube housing 1333 by an adhesive, an epoxy, a crimp, a weld, a friction fit, a set-screw, etc.

In one or more embodiments, an assembled reusable steerable handle may comprise an assembled reusable steerable handle with straight housing tube 1300 when control mechanism 1230 is extended in control mechanism guide 1255 relative to steerable handle base proximal end 1252, e.g., an assembled reusable steerable handle may comprise an assembled reusable steerable handle with straight housing tube 1300 when control mechanism 1230 is fully extended in control mechanism guide 1255 relative to steerable handle base proximal end 1252. Illustratively, an assembled reusable steerable handle may comprise an assembled reusable steerable handle with straight housing tube 1300 when control mechanism distal end 1231 is adjacent to steerable nosecone proximal end 1222, e.g., an assembled reusable steerable handle may comprise an assembled reusable steerable handle with straight housing tube 1300 when control mechanism distal end 1231 abuts steerable nosecone proximal end 1222. In one or more embodiments, an assembled reusable steerable handle may comprise an assembled reusable steerable handle with straight housing tube 1300 when optic fiber support mechanism 1100 is extended relative to housing tube 1260, e.g., an assembled reusable steerable handle may comprise an assembled reusable steerable handle with straight housing tube 1300 when optic fiber support mechanism 1100 is fully extended relative to housing tube 1260. Illustratively, an assembled reusable steerable handle may comprise an assembled reusable steerable handle with straight housing tube 1300 when housing tube distal end 1261 is adjacent to optic fiber support mechanism distal end 1101, e.g., an assembled reusable steerable handle may comprise an assembled reusable steerable handle with straight housing tube 1300 when housing tube distal end 1261 is coplanar with optic fiber support mechanism distal end 1101. In one or more embodiments, an assembled reusable steerable handle may comprise an assembled reusable steerable handle with straight housing tube 1300 when curved portion 1265 is disposed between anterior portion distal end 1111 and anterior portion proximal end 1112.

Illustratively, an actuation of control mechanism 1230 in control mechanism guide 1255 may be configured to retract control mechanism 1230 relative to steerable handle base 1250, e.g., an actuation of control mechanism proximal end 1232 towards steerable handle base proximal end 1252 may be configured to retract control mechanism 1230 relative to handle base 1250. In one or more embodiments, a retraction of control mechanism 1230 relative to steerable handle base 1250 may be configured to retract piston tube 1210 relative to steerable handle base 1250, e.g., an actuation of control mechanism proximal end 1232 towards steerable handle base proximal end 1252 may be configured to actuate piston tube proximal end 1212 towards steerable handle base proximal end 1252. Illustratively, a retraction of piston tube 1210 relative to steerable handle base 1250 may be configured to retract optic fiber support mechanism 1100 relative to steerable handle base 1250, e.g., an actuation of piston tube proximal end 1212 towards steerable handle base proximal end 1252 may be configured to actuate optic fiber support mechanism proximal end 1102 towards steerable handle base proximal end 1252. In one or more embodiments, a retraction of optic fiber support mechanism 1100 relative to steerable handle base 1250 may be configured to retract optic fiber support mechanism 1100 relative to housing tube 1260, e.g., a retraction of optic fiber support mechanism 1100 relative to steerable handle base 1250 may be configured to retract optic fiber support mechanism 1100 relative to housing tube 1260 wherein optic fiber support mechanism distal end 1101 is disposed between housing tube distal end 1261 and housing tube proximal end 1262. In one or more embodiments, a retraction of optic fiber support mechanism 1100 relative to housing tube 1260 may be configured to retract optic fiber support mechanism 1100 relative to curved portion 1265. Illustratively, a retraction of optic fiber support mechanism 1100 relative to curved portion 1265 may be configured to gradually curve housing tube 1260, e.g., a retraction of optic fiber support mechanism 1100 relative to curved portion 1265 may be configured to gradually curve housing tube 1260 until an assembled reusable steerable handle may comprise an assembled reusable steerable handle with curved housing tube 1301.

In one or more embodiments, an assembled reusable steerable handle may comprise an assembled reusable steerable handle with curved housing tube 1301 when control mechanism 1230 is retracted in control mechanism guide 1255 relative to steerable handle base proximal end 1252, e.g., an assembled reusable steerable handle may comprise an assembled reusable steerable handle with curved housing tube 1301 when control mechanism 1230 is fully retracted in control mechanism guide 1255 relative to steerable handle base proximal end 1252. Illustratively, an assembled reusable steerable handle may comprise an assembled reusable steerable handle with curved housing tube 1301 when control mechanism proximal end 1232 is adjacent to piston tube proximal housing distal end 1241, e.g., an assembled reusable steerable handle may comprise an assembled reusable steerable handle with curved housing tube 1301 when control mechanism proximal end 1232 piston tube proximal housing distal end 1241. In one or more embodiments, an assembled reusable steerable handle may comprise an assembled reusable steerable handle with curved housing tube 1301 when optic fiber support mechanism 1100 is retracted relative to housing tube 1260, e.g., an assembled reusable steerable handle may comprise an assembled reusable steerable handle with curved housing tube 1301 when optic fiber support mechanism 1100 is fully retracted relative to housing tube 1260. Illustratively, an assembled reusable steerable handle may comprise an assembled reusable steerable handle with curved housing tube 1301 when housing tube distal end 1261 is extended from optic fiber support mechanism distal end 1101, e.g., an assembled reusable steerable handle may comprise an assembled reusable steerable handle with curved housing tube 1301 when housing tube distal end 1261 is fully extended from optic fiber support mechanism distal end 1101. In one or more embodiments, an assembled reusable steerable handle may comprise an assembled reusable steerable handle with curved housing tube 1301 when curved portion 1265 is extended from anterior portion distal end 1111, e.g., an assembled reusable steerable handle may comprise an assembled reusable steerable handle with curved housing tube 1301 when curved portion 1265 is fully extended from anterior portion distal end 1111.

Illustratively, an actuation of control mechanism 1230 in control mechanism guide 1255 may be configured to extend control mechanism 1230 relative to steerable handle base 1250, e.g., an actuation of control mechanism distal end 1231 towards steerable handle base distal end 1251 may be configured to extend control mechanism 1230 relative to handle base 1250. In one or more embodiments, an extension of control mechanism 1230 relative to steerable handle base 1250 may be configured to extend piston tube 1210 relative to steerable handle base 1250, e.g., an actuation of control mechanism distal end 1231 towards steerable handle base distal end 1251 may be configured to actuate piston tube distal end 1211 towards steerable handle base distal end 1251. Illustratively, an extension of piston tube 1210 relative to steerable handle base 1250 may be configured to extend optic fiber support mechanism 1100 relative to steerable handle base 1250, e.g., an actuation of piston tube distal end 1211 towards steerable handle base distal end 1251 may be configured to actuate optic fiber support mechanism distal end 1101 away from steerable handle base proximal end 1252. In one or more embodiments, an extension of optic fiber support mechanism 1100 relative to steerable handle base 1250 may be configured to extend optic fiber support mechanism 1100 relative to housing tube 1260, e.g., an extension of optic fiber support mechanism 1100 relative to steerable handle base 1250 may be configured to extend anterior portion 1110 relative to housing tube 1260. In one or more embodiments, an extension of optic fiber support mechanism 1100 relative to housing tube 1260 may be configured to extend optic fiber support mechanism 1100 relative to curved portion 1265, e.g., an extension of optic fiber support mechanism 1100 relative to housing tube 1260 may be configured to extend anterior portion 1110 relative to curved portion 1265. Illustratively, an extension of optic fiber support mechanism 1100 relative to curved portion 1265 may be configured to gradually straighten housing tube 1260, e.g., an extension of optic fiber support mechanism 1100 relative to curved portion 1265 may be configured to gradually straighten housing tube 1260 until an assembled reusable steerable handle may comprise an assembled reusable steerable handle with straight housing tube 1300.

FIGS. 14A an 14B are schematic diagrams illustrating an assembled reusable steerable laser probe with single-use optic fiber 1400. FIG. 14A illustrates a side view of an assembled reusable steerable laser probe with single-use optic fiber 1400. FIG. 14B illustrates a cross-sectional view in a sagittal plane of an assembled reusable steerable laser probe with single-use optic fiber 1400. Illustratively, an assembled reusable steerable laser probe with single-use optic fiber 1400 may comprise an assembled single-use optic fiber 300, an assembled optic fiber fixture 500, and an assembled reusable steerable handle with straight housing tube 1300. In one or more embodiments, an assembled reusable steerable laser probe with single-use optic fiber 1400 may comprise a first transitory connector 100 disposed in a portion of assembled optic fiber fixture 500 and a second transitory connector 100 disposed in apportion of assembled reusable steerable handle with straight housing tube 1300.

In one or more embodiments, an assembled reusable steerable laser probe with single-use optic fiber 1400 may comprise a first transitory connector 100 disposed in a portion of assembled optic fiber fixture 500 wherein optic fiber proximal end 212 is adjacent to assembled optic fiber fixture distal end 501. Illustratively, an assembled reusable steerable laser probe with single-use optic fiber 1400 may comprise a first transitory connector 100 disposed in a portion of assembled optic fiber fixture 500 wherein optic fiber proximal end 212 abuts assembled optic fiber fixture distal end 501. In one or more embodiments, an assembled reusable steerable laser probe with single-use optic fiber 1400 may comprise a first transitory connector 100 disposed in a portion of assembled optic fiber fixture 500 wherein optic fiber proximal end 212 is coplanar with assembled optic fiber fixture distal end 501. Illustratively, inserting a portion of optic fiber 210 into a portion of assembled optic fiber fixture 500 may be configured to ingress optic fiber proximal end 212 into first transitory connector housing 550. In one or more embodiments, inserting a portion of optic fiber 210 into a portion of assembled optic fiber fixture 500 may be configured to ingress optic fiber proximal end 212 into first transitory connector housing distal taper 545. Illustratively, first transitory connector housing distal taper 545 may be configured to guide an ingress of optic fiber 210 into fixture facilitating sleeve inner bore 540. In one or more embodiments, inserting a portion of optic fiber 210 into a portion of assembled optic fiber fixture 500 may be configured to ingress optic fiber proximal end 212 into fixture facilitating sleeve inner bore 540. Illustratively, inserting a portion of optic fiber 210 into a portion of assembled optic fiber fixture 500 may be configured to ingress optic fiber proximal end 212 into fixture facilitating sleeve housing 535. In one or more embodiments, fixture facilitating sleeve housing 535 may be configured to guide an ingress of optic fiber 210 into fixture base inner bore 530. Illustratively, inserting a portion of optic fiber 210 into a portion of assembled optic fiber fixture 500 may be configured to ingress optic fiber proximal end 212 into fixture base inner bore 530. In one or more embodiments, inserting a portion of optic fiber 210 into a portion of assembled optic fiber fixture 500 may be configured to ingress optic fiber proximal end 212 into machine connector proximal taper 444. Illustratively, machine connector proximal taper 444 may be configured to guide an ingress of optic fiber 210 into machine connector proximal inner lumen 525. In one or more embodiments, inserting a portion of optic fiber 210 into a portion of assembled optic fiber fixture 500 may be configured to ingress optic fiber proximal end 212 into machine connector proximal inner lumen 525. Illustratively, inserting a portion of optic fiber 210 into a portion of assembled optic fiber fixture 500 may be configured to ingress optic fiber proximal end 212 into inner lumen proximal taper 520. In one or more embodiments, inner lumen proximal taper 520 may be configured to guide an ingress of optic fiber into machine connector distal inner lumen 515. Illustratively, inserting a portion of optic fiber 210 into a portion of assembled optic fiber fixture 500 may be configured to ingress optic fiber proximal end 212 into machine connector distal inner lumen 515. In one or more embodiments, inserting a portion of optic fiber 210 into a portion of assembled optic fiber fixture 500 may be configured to ingress optic fiber proximal end 212 into inner lumen distal taper 510. Illustratively, inner lumen distal taper 510 may be configured to guide an ingress of optic fiber 210 into optic fiber proximal end guide 505. In one or more embodiments, inserting a portion of optic fiber 210 into a portion of assembled optic fiber fixture 500 may be configured to ingress optic fiber proximal end 212 into optic fiber proximal end guide 505.

Illustratively, inserting first transitory connector 100 into a portion of assembled optic fiber fixture 500 may be configured to ingress optic fiber 210 into optic fiber proximal end guide 505, e.g., inserting first transitory connector 100 into a portion of assembled optic fiber fixture 500 may be configured to ingress optic fiber 210 into optic fiber proximal end guide 505 wherein optic fiber proximal end 212 is coplanar with assembled optic fiber fixture distal end 501. In one or more embodiments, inserting first transitory connector distal end 101 into first transitory connector housing distal taper 545 may be configured to ingress optic fiber 210 into optic fiber proximal end guide 505, e.g., inserting first transitory connector distal end 101 into first transitory connector housing distal taper 545 may be configured to ingress optic fiber 210 into optic fiber proximal end guide 505 wherein optic fiber proximal end 212 is coplanar with assembled optic fiber fixture distal end 501. Illustratively, inserting first transitory connector nosecone base 120 into first transitory connector housing 550 may be configured to ingress optic fiber 210 into optic fiber proximal end guide 505, e.g., inserting first transitory connector nosecone base 120 into first transitory connector housing 550 may be configured to ingress optic fiber 210 into optic fiber proximal end guide 505 wherein optic fiber proximal end 212 is coplanar with assembled optic fiber fixture distal end 501. In one or more embodiments, inserting major diameter 105 and minor diameter 106 into first transitory connector housing threading 560 may be configured to ingress optic fiber 210 into optic fiber proximal end guide 505, e.g., inserting major diameter 105 and minor diameter 106 into first transitory connector housing threading 560 may be configured to ingress optic fiber 210 into optic fiber proximal end guide 505 wherein optic fiber proximal end 212 is coplanar with assembled optic fiber fixture distal end 501. Illustratively, rotating major diameter 105 and minor diameter 106 in first transitory connector housing threading 560 may be configured to ingress optic fiber 210 into optic fiber proximal end guide 505, e.g., rotating major diameter 105 and minor diameter 106 in first transitory connector housing threading 560 may be configured to ingress optic fiber 210 into optic fiber proximal end guide 505 wherein optic fiber proximal end 212 is coplanar with assembled optic fiber fixture distal end 501. In one or more embodiments, rotating major diameter 105 and minor diameter 106 in first transitory connector housing threading 560 may be configured to fix first transitory connector 100 in assembled optic fiber fixture 500, e.g., rotating major diameter 105 and minor diameter 106 in first transitory connector housing threading 560 may be configured to fix major diameter 105 and minor diameter 106 in first transitory connector housing threading 560. For example, major diameter 105 and minor diameter 106 may comprise a threaded fastener.

Illustratively, inserting a portion of optic fiber 210 into a portion of assembled reusable steerable handle with straight housing tube 1300 may be configured to ingress optic fiber distal end 211 into second transitory connector housing 1320. In one or more embodiments, inserting a portion of optic fiber 210 into a portion of assembled reusable steerable handle with straight housing tube 1300 may be configured to ingress optic fiber distal end 211 into second transitory connector housing distal taper 1325. Illustratively, second transitory connector housing distal taper 1325 may be configured to guide an ingress of optic fiber 210 into steerable handle inner bore 1330. In one or more embodiments, inserting a portion of optic fiber 210 into a portion of assembled reusable steerable handle with straight housing tube 1300 may be configured to ingress optic fiber distal end 211 into steerable handle inner bore 1330. Illustratively, inserting a portion of optic fiber 210 into a portion of assembled reusable steerable handle with straight housing tube 1300 may be configured to ingress optic fiber distal end 211 into steerable handle inverted taper 1331. In one or more embodiments, inserting a portion of optic fiber 210 into a portion of assembled reusable steerable handle with straight housing tube 1300 may be configured to ingress optic fiber distal end 211 into steerable handle inner taper 1332. Illustratively, steerable handle inner taper 1332 may be configured to guide an ingress of optic fiber 210 into housing tube housing 1333. In one or more embodiments, inserting a portion of optic fiber 210 into a portion of assembled reusable steerable handle with straight housing tube 1300 may be configured to ingress optic fiber distal end 211 into housing tube housing 1333. Illustratively, inserting a portion of optic fiber 210 into a portion of assembled reusable steerable handle with straight housing tube 1300 may be configured to ingress optic fiber distal end 211 into housing tube 1260, e.g., inserting a portion of optic fiber 210 into a portion of assembled reusable steerable handle with straight housing tube 1300 may be configured to ingress optic fiber distal end 211 into housing tube proximal end 1262.

Illustratively, inserting second transitory connector 100 into a portion of assembled reusable steerable handle with straight housing tube 1300 may be configured to ingress optic fiber 210 into housing tube 1260, e.g., inserting second transitory connector 100 into a portion of assembled reusable steerable handle with straight housing tube 1300 may be configured to ingress optic fiber 210 into housing tube 1260 wherein optic fiber distal end 211 is adjacent to housing tube distal end 1261. In one or more embodiments, inserting second transitory connector 100 into a portion of assembled reusable steerable handle with straight housing tube 1300 may be configured to ingress optic fiber 210 into housing tube 1260 wherein optic fiber distal end 211 is coplanar with housing tube distal end 1261. Illustratively, inserting second transitory connector distal end 101 into second transitory connector housing distal taper 1325 may be configured to ingress optic fiber 210 into housing tube 1260, e.g., inserting second transitory connector distal end 101 into second transitory connector housing distal taper 1325 may be configured to ingress optic fiber 210 into housing tube 1260 wherein optic fiber distal end 211 is adjacent to housing tube distal end 1261. In one or more embodiments, inserting second transitory connector distal end 101 into second transitory connector housing distal taper 1325 may be configured to ingress optic fiber 210 into housing tube 1260 wherein optic fiber distal end 211 is coplanar with housing tube distal end 1261. Illustratively, inserting second transitory connector nosecone base 120 into second transitory connector housing 1320 may be configured to ingress optic fiber 210 into housing tube 1260, e.g., inserting second transitory connector nosecone base 120 into second transitory connector housing 1320 may be configured to ingress optic fiber 210 into housing tube 1260 wherein optic fiber distal end 211 is adjacent to housing tube distal end 1261. In one or more embodiments, inserting second transitory connector nosecone base 120 into second transitory connector housing 1320 may be configured to ingress optic fiber 210 into housing tube 1260 wherein optic fiber distal end 211 is coplanar with housing tube distal end 1261. Illustratively, inserting major diameter 105 in major diameter housing 1305 may be configured to ingress optic fiber 210 into housing tube 1260, e.g., inserting major diameter 105 in major diameter housing 1305 may be configured to ingress optic fiber 210 into housing tube 1260 wherein optic fiber distal end 211 is adjacent to housing tube distal end 1261. In one or more embodiments, inserting major diameter 105 in major diameter housing 1305 may be configured to ingress optic fiber 210 into housing tube 1260 wherein optic fiber distal end 211 is coplanar with housing tube distal end 1261. Illustratively, inserting minor diameter 106 into minor diameter housing 1306 may be configured to ingress optic fiber 210 into housing tube 1260, e.g., inserting minor diameter 106 into minor diameter housing 1306 may be configured to ingress optic fiber 210 into housing tube 1260 wherein optic fiber distal end 211 is adjacent to housing tube distal end 1261. In one or more embodiments, inserting minor diameter 106 into minor diameter housing 1306 may be configured to ingress optic fiber 210 into housing tube 1260 wherein optic fiber distal end 211 is coplanar with housing tube distal end 1261. Illustratively, rotating major diameter 105 in major diameter housing 1305 may be configured to ingress optic fiber 210 into housing tube 1260, e.g., rotating major diameter 105 in major diameter housing 1305 may be configured to ingress optic fiber 210 into housing tube 1260 wherein optic fiber distal end 211 is adjacent to housing tube distal end 1261. In one or more embodiments, rotating major diameter 105 in major diameter housing 1305 may be configured to ingress optic fiber 210 into housing tube 1260 wherein optic fiber distal end 211 is coplanar with housing tube distal end 1261. Illustratively, rotating minor diameter 106 in minor diameter housing 1306 may be configured to ingress optic fiber 210 into housing tube 1260, e.g., rotating minor diameter 106 in minor diameter housing 1306 may be configured to ingress optic fiber 210 into housing tube 1260 wherein optic fiber distal end 211 is adjacent to housing tube distal end 1261. In one or more embodiments, rotating minor diameter 106 in minor diameter housing 1306 may be configured to ingress optic fiber 210 into housing tube 1260 wherein optic fiber distal end 211 is coplanar with housing tube distal end 1261. Illustratively, rotating major diameter 105 in major diameter housing 1305 may be configured to fix second transitory connector 100 in assembled reusable steerable handle with straight housing tube 1300, e.g., rotating major diameter 105 in major diameter housing 1305 may be configured to fix major diameter 105 in major diameter housing 1305. In one or more embodiments, rotating minor diameter 106 in minor diameter housing 1306 may be configured to fix second transitory connector 100 in assembled reusable steerable handle with straight housing tube 1300, e.g., rotating minor diameter 106 in minor diameter housing 1306 may be configured to fix minor diameter 106 in minor diameter housing 1306. For example, major diameter 105 and minor diameter 106 may comprise a threaded fastener.

Illustratively, a user may perform a photocoagulation procedure with an assembled reusable steerable laser probe with single-use optic fiber 1400. In one or more embodiments, a user may connect machine connector 440 to a laser machine. Illustratively, a user may energize the laser machine to deliver laser light into optic fiber proximal end 212, through optic fiber 210, out from optic fiber distal end 211, and onto a surgical target site. In one or more embodiments, a retraction of control mechanism 1230 relative to steerable handle base 1250 may be configured to curve optic fiber 210. Illustratively, an extension of control mechanism 1230 relative to steerable handle base 1250 may be configured to straighten optic fiber 210. In one or more embodiments, assembled reusable steerable handle with straight housing tube 1300 may be a reusable medical device sold non-sterile and sterilized by a user in a medical autoclave.

The foregoing description has been directed to particular embodiments of this invention. It will be apparent; however, that other variations and modifications may be made to the described embodiments, with the attainment of some or all of their advantages. Specifically, it should be noted that the principles of the present invention may be implemented in any system. Furthermore, while this description has been written in terms of a laser probe, the teachings of the present invention are equally suitable to any systems where the functionality may be employed. Therefore, it is the object of the appended claims to cover all such variations and modifications as come within the true spirit and scope of the invention.

What is claimed is:

1. An instrument comprising:
a first transitory connector having a first transitory connector distal end and a first transitory connector proximal end;
a first inner bore of the first transitory connector;
a first inner bore distal taper of the first transitory connector;
a first optic fiber housing of the first transitory connector;
a second transitory connector having a second transitory connector distal end and a second transitory connector proximal end;
a second inner bore of the second transitory connector;
a second inner bore distal taper of the second transitory connector;
a second optic fiber housing of the second transitory connector;
a jacketing having a jacketing distal end and a jacketing proximal end wherein the jacketing distal end is disposed in the first inner bore and wherein the jacketing proximal end is disposed in the second inner bore; and
an optic fiber having an optic fiber distal end and an optic fiber proximal end, the optic fiber disposed in the jacketing, the first inner bore, the first inner bore distal taper, the first optic fiber housing, the second inner bore, the second inner bore distal taper, and the second optic fiber housing wherein a first portion of the optic fiber is fixed in the first optic fiber housing and a second portion of the optic fiber is fixed in the second optic fiber housing and wherein the optic fiber distal end extends a first distance from the first transitory connector distal end and the optic fiber proximal end extends a second distance from the second transitory connector proximal end and wherein the first distance is equal to the second distance.

2. The instrument of claim 1 further comprising:
a major diameter of the first transitory connector.

3. The instrument of claim 2 further comprising:
a minor diameter of the first transitory connector.

4. The instrument of claim 1 further comprising:
a handle facilitating sleeve having a handle facilitating sleeve distal end and a handle facilitating sleeve proximal end;
a transitory connector housing of the handle facilitating sleeve; and
a handle facilitating sleeve inner bore of the handle facilitating sleeve.

5. Thee instrument of claim 4 wherein the handle facilitating sleeve and the optic fiber have a coefficient of friction in a range of 0.011 to 0.36.

6. The instrument of claim 4 wherein the handle facilitating sleeve and the optic fiber have a coefficient of friction of less than 0.011.

7. The instrument of claim 4 wherein the handle facilitating sleeve is manufactured from a fluorocarbon material.

8. The instrument of claim 4 wherein the handle facilitating sleeve is manufactured from a material having a density in a range of 0.024 to 0.073 pounds per cubic inch.

9. The instrument of claim 4 wherein the handle facilitating sleeve is manufactured from a self-lubricating thermoplastic material.

10. The instrument of claim 4 wherein the handle facilitating sleeve is manufactured from a turcite material.

11. The instrument of claim 4 wherein the handle facilitating sleeve is manufactured from a material having a hardness in a range of 50 Shore D to 75 Shore D.

12. he instrument of claim 4 wherein the handle facilitating sleeve is manufactured from a material having a first hardness and the optic fiber is manufactured from a material having a second hardness wherein the second hardness is greater than the first hardness.

13. The instrument of claim 4 further comprising:
a handle base having a handle base distal end and a handle base proximal end; and
a handle facilitating sleeve housing of the handle base.

14. The instrument of claim 13 wherein the handle facilitating sleeve is disposed in the handle facilitating sleeve housing.

15. The instrument of claim 14 further comprising:
a handle nosecone having a handle nosecone distal end and a handle nosecone proximal end; and
a hypodermic tube housing of the handle nosecone.

16. The instrument of claim 15 further comprising:
a nosecone housing of the handle base wherein the handle nosecone is disposed in the nosecone housing.

17. The instrument of claim 16 further comprising:
a hypodermic tube having a hypodermic tube distal end and a hypodermic tube proximal end.

18. The instrument of claim 17 further comprising:
a hypodermic tube distal taper of the hypodermic tube.

19. e instrument of claim 17 further comprising:
a hypodermic tube proximal taper of the hypodermic tube.

20. The instrument of claim 17 wherein the hypodermic tube is disposed in the hypodermic tube housing and wherein the optic fiber is disposed in the handle facilitating sleeve and the hypodermic tube.
